(19) United States
(12) Patent Application Publication
(10) Pub. No.: US 2023/0226158 A1
(43) Pub. Date: Jul. 20, 2023

(54) COMPOSITIONS AND METHODS FOR TREATING AND/OR PREVENTING COAGULOPATHY AND/OR SEPSIS IN PATIENTS SUFFERING FROM BACTERIAL AND/OR VIRAL INFECTIONS

(71) Applicant: YALE UNIVERSITY, New Haven, CT (US)

(72) Inventors: Paul Stabach, New Haven, CT (US); Demetrios Braddock, New Haven, CT (US)

(21) Appl. No.: 18/000,957

(22) PCT Filed: Jun. 8, 2021
(86) PCT No.: PCT/US2021/036368

§ 371 Date: Dec. 7, 2022

Related U.S. Application Data
(60) Provisional application No. 63/035,956, filed on Jun. 8, 2020.

(51) Int. Cl. A61K 38/46; A61P 31/04

(52) U.S. Cl. CPC A61K 38/465; A61P 31/04

(57) ABSTRACT

The present disclosure includes compositions and methods for treating, ameliorating, and/or preventing immune mediated pathology associated with a bacterial and/or viral infection.

### CROSS-REFERENCE TO RELATED APPLICATIONS

This application claims priority under 35 U.S.C. § 119(e) to U.S. Provisional Patent Application No. 63/035,956, filed Jun. 8, 2020, which is hereby incorporated herein by reference in its entirety.

### SEQUENCE LISTING

The ASCII text file named “047162-7288WO1(01380) Sequence Listing ST25” created on Jun. 6, 2021, comprising 160 Kbytes, is hereby incorporated by reference in its entirety.

### BACKGROUND

Coronavirus disease 2019 (COVID-19) is an infectious disease caused by a recently isolated virus known as severe acute respiratory syndrome coronavirus 2 (SARS-CoV-2). COVID-19 is an ongoing global pandemic, which has sickened about 4.5 million people and killed more than 300,000 people worldwide. Currently there are no available vaccines or antiviral treatments for the treatment or prevention of COVID-19.

Common symptoms of COVID-19 include fever, cough, fatigue, shortness of breath, and loss of smell and taste. Most COVID-19 infections result in mild symptoms and resolve on their own, but some cases progress to acute respiratory distress syndrome (ARDS), pneumonia, multi-organ failure, systemic inflammation, septic shock, heart failure, arrhythmias, and blood clots, and eventually death.

There is thus a need in the art to identify therapeutic agents and treatments that can be used to treat or prevent complications from COVID-19 in an infected subject. The present disclosure addresses and meets this need.

### BRIEF SUMMARY

The disclosure provides a method of treating, ameliorating, and/or preventing inefficient NET hydrolysis (“NETolysis”) in a subject afflicted with a bacterial and/or viral infection.

The disclosure provides a method of treating, ameliorating, and/or preventing systemic inflammation, organ damage, and/or sepsis in a subject afflicted with a bacterial and/or viral infection.

The disclosure provides a method of treating, ameliorating, and/or preventing pathologic thrombosis in a subject afflicted with a bacterial and/or viral infection.

In certain embodiments, the method comprises administering to the subject a therapeutically effective amount of a construct comprising the amino acid sequence:

Y—X1-LINKER-Fc-X2 (I)

wherein Y, X1, LINKER, X2, and Fc are defined elsewhere herein.

### BRIEF DESCRIPTION OF THE DRAWINGS

The following detailed description of illustrative embodiments of the disclosure will be better understood when read in conjunction with the appended drawings. For the purpose of illustrating the disclosure, exemplary embodiments are shown in the drawings. It should be understood, however, that the disclosure is not limited to the precise arrangements and instrumentalities of the embodiments shown in the drawings.

FIG. 1 illustrates neutrophil extracellular trap (NET) formation. Scanning electron microscopy of neutrophil (marked as A) casting a net (marked as B) entrapping Helicobacter pylori bacteria (some of which are marked as C). Image taken from Kumamoto T, et al., 2006, Eur. Heart J. 27(17):2081-7.

FIG. 2 illustrates a non-limiting DNAse1-Fc construct of the disclosure, with certain contemplated point mutations highlighted.

FIG. 3 illustrates a non-limiting DNAse1L3-Fc construct of the disclosure, with certain contemplated point mutations highlighted.

FIG. 4 illustrates a non-limiting DNAse1-Fc construct of the disclosure, with certain contemplated point mutations highlighted.

FIG. 5 illustrates non-limiting constructs of the disclosure, with certain contemplated point mutations highlighted. In certain embodiments, certain mutations render the rDNAse hyperactive and/or render the rDNAse actin-resistant (i.e., has decreased affinity for actin) and/or increase the construct's half-life. The non-limiting aligment of amino acid sequences of mouse DNAse1 (SEQ ID NO:42) and mouse DNAse1L3 (SEQ ID NO:43) is illustrated.

FIG. 6 illustrates non-limiting constructs of the disclosure, with certain contemplated point mutations highlighted. In certain embodiments, the construct lacks at least a portion of the DNAse1L3 nuclear localization domain.

FIG. 7 illustrates a gel indicating that certain DNAse1L3 clones cleave chromatin, but that is not the case for certain DNAse1 clones.

FIG. 8 illustrates a non-limiting construct of the disclosure. In certain embodiments, the DNAse1 polypeptide is fused with the C-terminus tail of DNAse1L3.

FIGS. 9A-9D illustrate certain aspects of production and purification of DNAse-Fc constructs.

FIG. 10 illustrates a non-limiting enzyme optimization pathway to be applied to NET degrading enzymes, as illustrated with an exemplary protein and/or polypeptide.

FIGS. 11A-11D illustrate selected results for optimization of NET degrading enzymes. FIG. 11A: Free (or plasmid) DNA in the blood was degraded by DNAse1. Histone associated DNA was degraded by DNAse1L3. FIG. 11B: PK Assay of optimized DNAse1 and DNAse1L3 constructs. Mice were injected with 1 mg/kg biologic; blood was withdrawn at various time points; exogenous plasmid or histone associated DNA is added; samples were incubated for 15 min.; degradation of DNA determined by agarose gels. FIGS. 11C-11D: PK of enzyme biologics determined in mice. Lanes 1-2: construct 1171; Lanes 3-4: construct 1671; Lanes 5-6: construct 1687; Lanes 7-8: Mock Injection. Constructs 1671 and 1687 readily degraded both plasmid (top) and chromatin DNA at 91 hours (FIG. 11C), and the activity persisted for 257 hours (FIG. 11D).

FIG. 12 illustrates certain aspects of production and purification of DNAse-Fc constructs.

### DETAILED DESCRIPTION OF THE DISCLOSURE

The present disclosure relates, in one aspect, to the discovery of certain constructs, compositions, and methods for treating, ameliorating, and/or preventing immune mediated pathology associated with a bacterial and/or viral infection.

In certain embodiments, the constructs contemplated herein can be used to treat, ameliorate, and/or prevent inefficient neutrophilic extracellular trap (“NET”) hydrolysis (“NETolysis”) in subject afflicted with a bacterial and/or viral infection.

In certain embodiments, the virus is a coronavirus. In other embodiments, the coronavirus is SARS-Cov and/or SARS-Cov-2.

Polymorphic neutrophils (PMNs) are the most abundant white blood cells in blood. PMNs circulate in tissues and blood, where they seek out invading micro-organisms. Upon encountering micro-organisms, the PMNs respond with an array of mechanisms to combat the infection, which include phagocytosis and/or release of stored antimicrobial compounds in a process called degranulation. In response to overwhelming infections, PMNs can self-destruct, extruding entrapping DNA and cytotoxic material forming what is known as “neutrophilic extracellular traps” (NETs). NETs trap invading pathogens in a sticky chromatin web, attached to which are an assortment of antimicrobial cytotoxic proteins and peptides released along with the chromatin when PMN degranulate in response to infectious stimuli. The high concentration of antimicrobial compounds maintained by NETs in close proximity to invading organisms increases the potency of the cytotoxic agents in an attempt to neutralize the pathogen and prevent its spread.

NETs are essential to the innate immune response. NETs are typically degraded by blood-based metallo-enzymes, and several circulating enzyme isoforms hydrolyze the high energy bonds in DNA to induce “NETolysis.” To do so, these enzymes recognize DNA as either free nucleic acid or in association with proteins such as the chromatin in the protein backbone of NETs. While NETs play an important role in the immune system, they must also be cleared quickly and efficiently, as failure to do so has serious pathologic consequences.

Overwhelming or uncontrolled “NETosis” may lead to systemic inflammation, coagulopathies, and/or remote organ failures. In certain embodiments, inefficient NET degradation is a central immune mediated mechanism responsible for the morbidity and mortality of COVID-19 infection. In fact, disseminated intravascular coagulation (DIC), thrombosis, and sepsis are significantly associated with mortality in severe confirmed COVID-19 cases. Moreover, in severely affected COVID-19 patients a ‘Sequential Organ Failure Assessment’ score of 5.65 (P<0.0001) is associated with dramatically increased D-dimers (>1 μg/mL), sepsis, and mortality, directly linking coagulopathy to organ ischemia as an end product of the immune mediated pathology associated with COVID-19 infection.

The importance of NETs in COVID-19 morbidity is supported by animal models and human clinical data on sepsis. Neutrophil infiltration is a key mediator of organ dysfunction through the production of reactive oxygen and nitrogen species in the vital organs during sepsis, and NETs are directly implicated in septic organ dysfunction in infants and adults. In septic infant mice, the observed high levels of NETs are due to the overwhelming NET production by neutrophils, and NETs correlate directly with organ failure and severity of sepsis. Further, NETs contain trapped histones, which are an established mediator of endothelial dysfunction, organ failure, and death in septic patients.

The present disclosure relates to central mechanisms of immune mediated pathology responsible for the morbidity associated with COVID-19 infection and presents enhanced NET degradation as an interventional agent in the pathogenic response. In certain embodiments, COVID-19 infection induces an immune mediated neutrophilic response resulting in excessive NET formation, leading to clinical sepsis, vasculopathy, DIC, and organ failure in severely infected patients. Indeed, DIC and thrombosis correlates inversely with COVID-19 survival, and a coagulopathy consistent with excessive NET formation is observed in critically ill COVID-19 patients.

The present disclosure provides bioavailable NET degrading constructs, which are capable of hydrolyzing NETs in vivo and can be used to treat the subjects afflicted with the diseases and/or disorders contemplated herein. Homeostatically, NETs are cleared from circulation by a process of hydrolysis mediated by the blood-based metallo-enzymes DNAse1 and DNAse1L3. In certain embodiments, the constructs contemplated within the disclosure have improved stability and/or bioavailability over the naturally occurring enzymes. In other embodiments, the constructs contemplated within the disclosure are useful in treating, ameliorating, and/or preventing immune mediated pathology driven by inefficient NET degradation in bacterial and/or viral infection. In yet other embodiments, the constructs contemplated within the disclosure are useful to prevent and/or ameliorate poor outcomes in bacterial and/or viral infection.

The present disclosure provides stable and bioavailable constructs comprising DNAse1L3 and/or DNAse1 polypeptides (or fragments, rearrangements, (point) mutations, truncations, and/or any other modifications and/or analogues and/or derivatives thereof) fused with certain proteins. In certain embodiments, the constructs contemplated herein have increased bioavailability and/or developability over the DNAse1L3 and/or DNAse1 polypeptides known in the art. In certain embodiments, the constructs contemplated herein have enhanced enzymatic activity over the DNAse1L3 and/or DNAse1 polypeptides known in the art. In certain embodiments, the constructs contemplated herein have improved pharmacokinetic behavior over the DNAse1L3 and/or DNAse1 polypeptides known in the art. In certain embodiments, the constructs contemplated herein have enhanced stability over the DNAse1L3 and/or DNAse1 polypeptides known in the art.

In certain embodiments, the in vivo half-life of a construct of the disclosure is at least about 1.5, 2, 2.5, 3, 4, 5, 6, 7, 8, 9, 10, 12, 14, 16, 18, and/or 20 times higher than the DNAse1 and/or DNAse1L3 polypeptides described in the art. In other embodiments, the constructs of the disclosure are administered to the subject at a lower dose and/or at a lower frequency than other DNAse1 and/or DNAse1L3 polypeptides in the art. In yet other embodiments, the constructs of the disclosure are administered to the subject once a month, twice a month, three times a month, and/or four times a month. In yet other embodiments, the lower frequency administration of the constructs of the disclosure results in better patient compliance and/or increased efficacy as compared with other DNAse1 and/or DNAse1L3 polypeptides in the art.

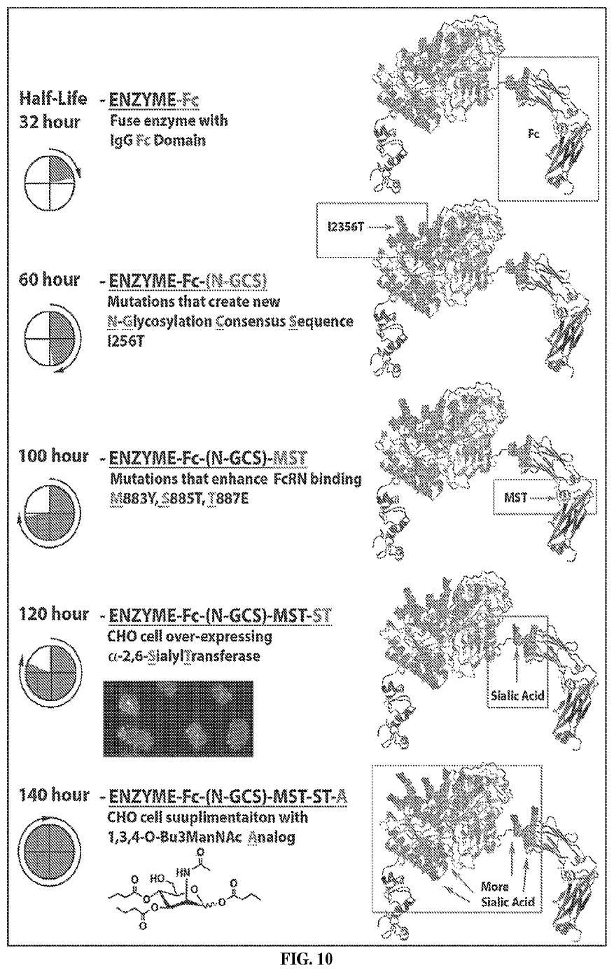

In one aspect, the present disclosure provides strategies for increasing the potency of enzyme biologics. In certain non-limiting embodiments, the present approach involves the stepwise improvement in the pharmacokinetic properties of a protein therapeutic by exploiting a suite of protein and glycosylation engineering methods. The approach is illustrated in FIG. 10. In certain embodiments, the present disclosure contemplates adding one or more N-glycans to the protein and/or polypeptide. In certain embodiments, the present disclosure contemplates optimizing pH-dependent cellular recycling of the protein and/or polypeptide by protein engineering of the Fc neonatal receptor (FcRn). In certain embodiments, the present disclosure contemplates improving sialylation of the protein and/or polypeptide by first producing ENPP1-Fc in cells stably transfected with human α-2,6-sialyltransferase (ST6). In certain embodiments, the present disclosure contemplates enhancing terminal sialylation of the protein and/or polypeptide by supplementing the production platform with 1,3,4-O-Bu₃ManNAc. Each of these steps can increased the area under the curve (AUC, a measure of in vivo drug availability) for the protein and/or polypeptide. In certain embodiments, this approach potentially extends once-a-day treatment to a monthly or bi-monthly dosing frequency.

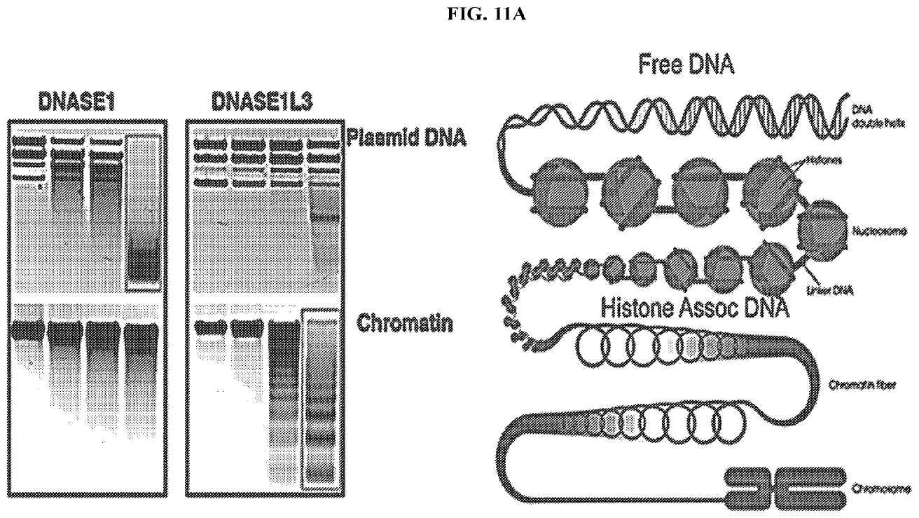

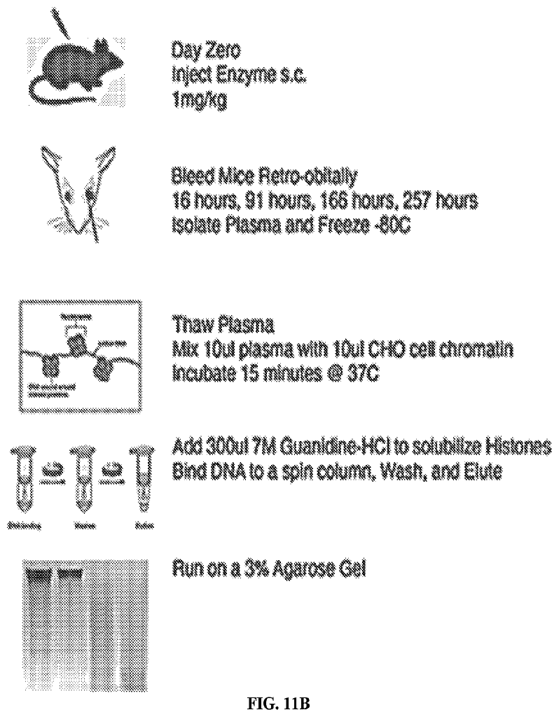

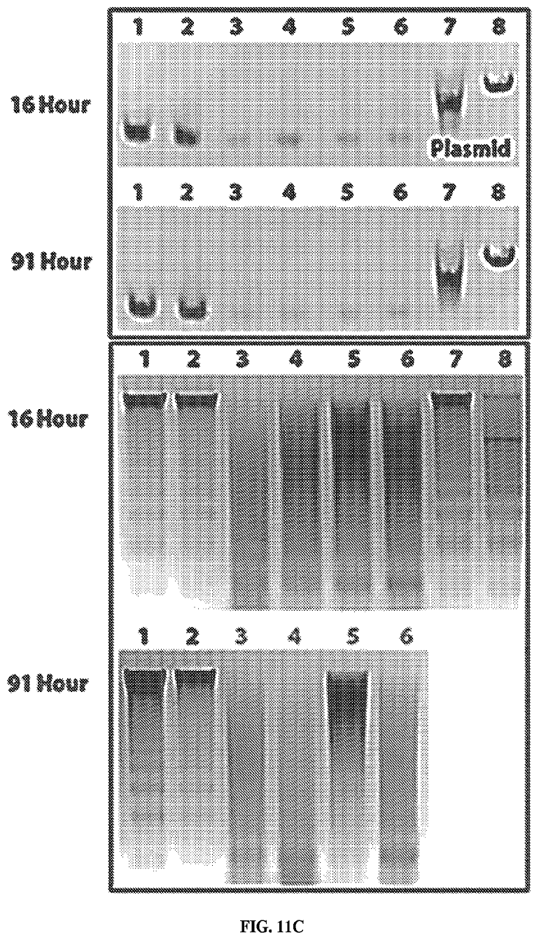

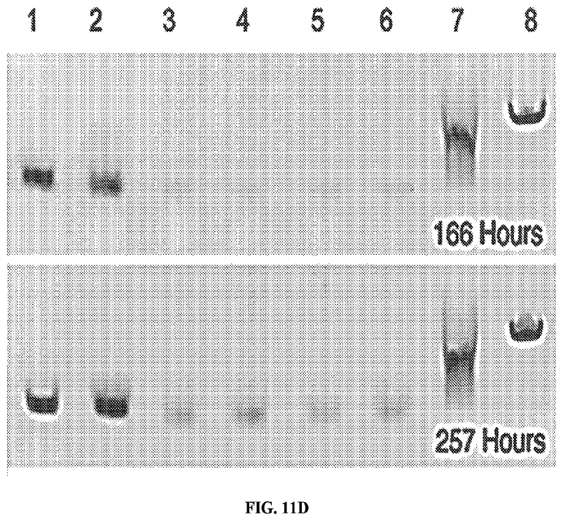

The methodology contemplated within the disclosure was applied to blood metalloenzymes known to degrade DNA—DNAse1 and DNas1L3. DNAse1 degrades free DNA in the plasma while DNAse1L3 degrades histone associated DNA (FIG. 11A). Using a combination of the techniques described elsewhere herein and other protein engineering methods, a construct that degrades both free and histone bound DNA in plasma was identified (FIG. 11C). This construct exhibits complete in vivo degradation of DNA in plasma for up to 257 hours following a single 1 mg/Kg subcutaneous injection (FIG. 11B-11D).

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, separation science, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of ±20% or ±10%, in certain embodiments ±5%, in certain embodiments ±1%, in certain embodiments ±0.1% from the specified value, as such variations are appropriate to perform the disclosed methods.

A disease or disorder is “alleviated” if the severity of a symptom of the disease or disorder, the frequency with which such a symptom is experienced by a patient, or both, is reduced.

As used herein the terms “alteration,” “defect,” “variation” or “mutation” refer to a mutation in a gene in a cell that affects the function, activity, expression (transcription or translation) or conformation of the polypeptide it encodes, including missense and nonsense mutations, insertions, deletions, frameshifts and premature terminations.

The term “antibody,” as used herein, refers to an immunoglobulin molecule that is able to specifically bind to a specific epitope on an antigen. Antibodies can be intact immunoglobulins derived from natural sources or from recombinant sources and can be immunoreactive portions of intact immunoglobulins.

As used herein, the term “AUC” refers to the area under the plasma drug concentration-time curve (AUC) and correlates with actual body exposure to drug after administration of a dose of the drug. In certain embodiments, the AUC is expressed in mg*h/L. The AUC can be used to measure bioavailability of a drug, which is the fraction of unchanged drug that is absorbed intact and reaches the site of action, or the systemic circulation following administration by any route.

AUC can be calculated used Linear Trapezoidal method or Logarithmic Trapezoidal method. The Linear Trapezoidal method uses linear interpolation between data points to calculate the AUC. This method is required by the OGD and FDA, and is the standard for bioequivalence trials. For a given time interval (t₁−t₂), the AUC can be calculated as follows:

$$A U C = \frac{1}{2} ( {C}_{1} + {C}_{2} ) ( {t}_{2} - {t}_{1} )$$

wherein C₁ and C₂ are the average concentration over the time interval (t₁ and t₂).

The Logarithmic Trapezoidal method uses logarithmic interpolation between data points to calculate the AUC. This method is more accurate when concentrations are decreasing because drug elimination is exponential (which makes it linear on a logarithmic scale). For a given time interval (t₁−t₂), the AUC can be calculated as follows (assuming that C₁>C₂):

$$A U C = \frac{{C}_{1} - {C}_{2}}{ln ( {C}_{1} ) - ln ( {C}_{2} )} ( {t}_{2} - {t}_{1} )$$

The term “bioavailability” as used herein refers to the extent and rate at which the active moiety (protein or drug or metabolite) enters systemic circulation, thereby accessing the site of action, or the systemic circulation following administration by any route. Bioavailability of an active moiety is largely determined by the properties of the dosage form, which depend partly on its design and manufacture. Differences in bioavailability among formulations of a given drug or protein can have clinical significance; thus, knowing whether drug formulations are equivalent is essential. The most reliable measure of a drug's or protein's bioavailability is area under the plasma concentration-time curve (AUC). AUC is directly proportional to the total amount of unchanged drug or therapeutic protein that reaches systemic circulation. Drug or therapeutic protein may be considered bioequivalent in extent and rate of absorption if their plasma concentration curves are essentially superimposable. For an intravenous dose of a drug, bioavailability is defined as unity. For drug administered by other routes of administration, bioavailability is often less than unity. Incomplete bioavailability may be due to a number of factors that can be subdivided into categories of dosage form effects, membrane effects, and site of administration effect. Half-life and AUC provide information about the bioavailability of a drug or biologic.

As used herein, the terms “conservative variation” or “conservative substitution” as used herein refers to the replacement of an amino acid residue by another, biologically similar residue. Conservative variations or substitutions are not likely to change the shape of the peptide chain. Examples of conservative variations, or substitutions, include the replacement of one hydrophobic residue such as isoleucine, valine, leucine or methionine for another, or the substitution of one polar residue for another, such as the substitution of arginine for lysine, glutamic for aspartic acid, or glutamine for asparagine.

As used herein, a “construct” of the disclosure refers to a fusion polypeptide comprising a DNAse1 and/or DNAse1L3 polypeptide, or any fragments, rearrangements, (point) mutations, truncations, or any other modifications and/or analogues and/or derivatives thereof.

A “disease” is a state of health of an animal wherein the animal cannot maintain homeostasis, and wherein if the disease is not ameliorated then the animal's health continues to deteriorate.

A “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “DNAse1” refers to deoxyribonuclease-1 (UniProtKB=P24855). The sequence of human DNAse1 is provided herein (SEQ ID NO:1). In certain embodiments, the signal peptide of DNAse1 corresponds to residues 1-22 of SEQ ID NO:1.

```
SEQ ID NO: 1
                10 20 30 40
MRGMKLLGAL LALAALLQGA VSLKIAAFNI QTFGETKMSN
                50 60 70 80
ATLVSYIVQI LSRYDIALVQ EVRDSHLTAV GKLLDNLNQD
                90 100 110 120
APDTYHYVVS EPLGRNSYKE RYLFVYRPDQ VSAVDSYYYD
              130 140 150 160
DGCEPCGNDT FNREPAIVRF FSRFTEVREF AIVPLHAAPG
              170 180 190 200
DAVAEIDALY DVYLDVQEKW GLEDVMLMGD FNAGCSYVRP
              210 220 230 240
SQWSSIRLWT SPTFQWLIPD SADTTATPTH CAYDRIVVAG
              250 260 270 280
MLLRGAVVPD SALPFNFQAA YGLSDQLAQA ISDHYPVEVM LK
```

The sequence of mouse DNAse1 is provided herein (SEQ ID NO:29):

MRYTGLMGTLLTLVNLLQLAGTLRIAAFNIRTFGETKMSNATLSVYFVK
ILSRYDIAVIQEVRDSHLVAVGKLLDELNRDKPDTYRYVVSEPLGRKSY
KEQYLFVYRPDQVSILDSYQYDDGCECGNDTFSREPAIVKFFSPYTEVQ
EFAIVPLHAAPTEAVSEIDALYDVYLDVWQKWGLEDIMFMGDFNAGCSY
VTSSQWSSIRLRTSPIFQWLIPDSADTTVTSTHCAYDRIVVAGALLQAA
VVPNSAVPFDFQAEYGLSNQLAEAISDHYPVEVTLRKI

The sequence alignment of human DNAse1 (SEQ ID NO:1, sequence ‘1’ below) and mouse DNAse1 (SEQ ID NO:29, sequence ‘2’ below) follows:

| DNAse1 | | |
|---|---|---|
| 1 | MRGMKLLGALLALAALLQGAVSLKIAAFNIQTFGETKMSNATLVSYIVQILSRYDIALVQ | 60 |
| 2 | MRYTGLMGTLLTLVNLLQLAGTLRIAAFNIRTFGETKMSNATLSVYFVKILSRYDIAVIQ | 60 |
| | ** *:*:**:*. *** * :*:******:************ *:*:********::* | |
| 1 | EVRDSHLTAVGKLLDNLNQDAPDTYHYVVSEPLGRNSYKERYLFVYRPDQVSAVDSYYYD | 120 |
| 2 | EVRDSHLVAVGKLLDELNRDKPDTYRYVVSEPLGRKSYKEQYLFVYRPDQVSILDSYQYD | 120 |
| | *******.*******:**:* ****:*********:****:*********** :*** ** | |
| 1 | DGCEPCGNDTFNREPAIVRFFSRFTEVREFAIVPLHAAPGDAVAEIDALYDVYLDVQEKW | 180 |
| 2 | DGCE-CGNDTFSREPAIVKFFSPYTEVQEFAIVPLHAAPTEAVSEIDALYDVYLDVWQKW | 179 |
| | **** ******.******:*** :***:*********** :**:************ :** | |
| 1 | GLEDVMLMGDFNAGCSYVRPSQWSSIRLWTSPTFQWLIPDSADTTATPTHCAYDRIVVAG | 240 |
| 2 | GLEDIMFMGDFNAGCSYVTSSQWSSIRLRTSPIFQWLIPDSADTTVTSTHCAYDRIVVAG | 239 |
| | ****:*:*********** ******** *** ************.* ************ | |
| 1 | MLLRGAVVPDSALPFNFQAAYGLSDQLAQAISDHYPVEVMLK-- | 282 |
| 2 | ALLQAAVVPNSAVPFDFQAEYGLSNQLAEAISDHYPVEVTLRKI | 283 |
| | **:.****:**:**:*** ****:***:********** *: | |

As used herein, “human DNAse1” refers to the human DNAse1 sequence as described herein, or any fragments, rearrangements, (point) mutations, truncations, or any other modifications and/or analogues and/or derivatives thereof. As used herein, the term “enzymatically active” with respect to DNAse1 is defined as being capable of binding and hydrolyzing DNA.

As used herein, the term “DNAse1L3” refers to deoxyribonuclease gamma (UniProtKB=Q13609). The sequence of human DNAse1L3 is provided herein (SEQ ID NO:2). In certain embodiments, the signal peptide of DNAse1L3 corresponds to residues 1-20 of SEQ ID NO:2. In certain embodiments, the nuclear localization signal of DNAse1L3 corresponds to residues 296-304 of SEQ ID NO:2. In certain embodiments, the nuclear localization signal of DNAse1L3 corresponds to residues 292-304 of SEQ ID NO:2. In certain embodiments, the nuclear localization signal of DNAse1L3 corresponds to residues 291-305 of SEQ ID NO:2. In certain embodiments, the nuclear localization signal of DNAse1L3 corresponds to residues A-B of SEQ ID NO:2, wherein A ranges from 291 to 296 and B ranges from 304 to 305.

```
SEQ ID NO: 2
                10 20 30 40
MSRELAPLLL LLLSIHSALA MRICSFNVRS FGESKQEDKN
                50 60 70 80
AMDVIVKVIK RCDIILVMEI KDSNNRICPI LMEKLNRNSR
                90 100 110 120
RGITYNYVIS SRLGRNTYKE QYAFLYKEKL VSVKRSYHYH
              130 140 150 160
DYQDGDADVF SREPFVVWFQ SPHTAVKDFV IIPLHTTPET
              170 180 190 200
SVKEIDELVE VYTDVKHRWK AENFIFMGDF NAGCSYVPKK
              210 220 230 240
AWKNIRLRTD PRFVWLIGDQ EDTTVKKSTN CAYDRIVLRG
              250 260 270 280
QEIVSSVVPK SNSVFDFQKA YKLTEEEALD VSDHFPVEFK
              290 300
LQSSRAFTNS KKSVTLRKKT KSKRS
```

The sequence of mouse DNAse1L3 is provided herein (SEQ ID NO:30):

MSLHPASPRLASLLLEILALHDTLALRLCSFNVR
SFGASKKENHEAMDIIVKIIKRCDLILLMEIKDS
SNNICPMLMEKLNGNSRRSTTYNYVISSRLGRNT
YKEQYAFVYKEKLVSVKTKYHYHDYQDGDTDVFS
REPFVVWFHSPFTAVKDFVIVPLHTTPETSVKEI
DELVDVYTDVRSQWKTENFIFMGDFNAGCSYVPK
KAWQNIRLRTDPKFVWLIGDQEDTTVKKSTSCAY
DRIVLCGQEIVNSVVPRSSGVFDFQKAYDLSEEE
ALDVSDHFPVEFKLQSSRAFTNNRKSVSLKKRKK
GNRS

The sequence alignment of human DNAse1L3 (SEQ ID NO:2, sequence ‘1’ below) and mouse DNAse1L3 (SEQ ID NO:30, sequence ‘2’ below) follows:

| DNAse1L3 | | |
|---|---|---|
| 1 | -----MSRELAPLLLLLLSIHSALAMRICSFNVRSFGESKQEDKNAMDVIVKVIKRCDII | 55 |
| 2 | MSLHPASPRLASLLLFILALHDTLALRLCSFNVRSFGASKKENHEAMDIIVKIIKRCDLI | 60 |
| | *.** ***::*::*.:**:*:********** *:*:::***:***:*****:* | |
| 1 | LVMEIKDSNNRICPILMEKLNRNSRRGITYNYVISSRLGRKTYKEQYAFLYKEKLVSVKR | 115 |
| 2 | LLMEIKDSSNNICPMLMEKLNGNSRRSTTYNYVISSRLGRNTYKEQYAFVYKEKLVSVKT | 120 |
| | *:******.*.***:**********. ************:********:********* | |
| 1 | SYHYHDYQDGDADVFSREPFVVWFQSPHTAVKDFVIIPLHTTPETSVKEIDELVEVYTDV | 175 |
| 2 | KYHYHDYQDGDTDVFSREPFVVWFHSPFTAVKDFVIVPLHTTPETSVKEIDELVDVYTDV | 180 |
| | .**********:************:**.********:*****************:***** | |
| 1 | KHRWKAENFIFMGDFNAGCSYVPKKAWKNIRLRTDPRFVWLIGDQEDTTVKKSTNCAYDR | 235 |
| 2 | RSQWKTENFIFMGDFNAGCSYVPKKAWQNIRLRTDPKFVWLIGDQEDTTVKKSTSCAYDR | 240 |
| | : :**:*********************:********:*****************.***** | |
| 1 | IVLRGQEIVSSVVPKSNSVFDFQKAYKLTEEEALDVSDHFPVEFKLQSSRAFTNSKKSVT | 295 |
| 2 | IVLCGQEIVNSVVPRSSGVFDFQKAYDLSEEEALDVSDHFPVEFKLQSSRAFTNNRKSVS | 300 |
| | *** *****.****:*..********.*:*************************.:***: | |
| 1 | LRKKTKSKRS | 305 |
| 2 | LKKRKKGNRS | 310 |
| | *:*:.*.:** | |

As used herein, “human DNAse1L3” refers to the human DNAse1L3 sequence as described herein, or any fragments, rearrangements, (point) mutations, truncations, or any other modifications and/or analogues and/or derivatives thereof. As used herein, the term “enzymatically active” with respect to DNAse1L3 is defined as being capable of binding and hydrolyzing DNA.

As used herein, the term “DNAse1-Fc” refers to a DNAse1 polypeptide recombinantly fused and/or chemically conjugated (including both covalent and non-covalent conjugations) to an FcR binding domain of an IgG molecule (preferably, a human IgG). In certain embodiments, the C-terminus of DNAse1 is fused or conjugated to the N-terminus of the FcR binding domain. In certain embodiments, the N-terminus of DNAse1 is fused or conjugated to the C-terminus of the FcR binding domain.

As used herein, the term “DNAse1L3-Fc” refers to a DNAse1L3 polypeptide recombinantly fused and/or chemically conjugated (including both covalent and non-covalent conjugations) to an FcR binding domain of an IgG molecule (preferably, a human IgG). In certain embodiments, the C-terminus of DNAse1L3 is fused or conjugated to the N-terminus of the FcR binding domain. In certain embodiments, the N-terminus of DNAse1L3 is fused or conjugated to the C-terminus of the FcR binding domain.

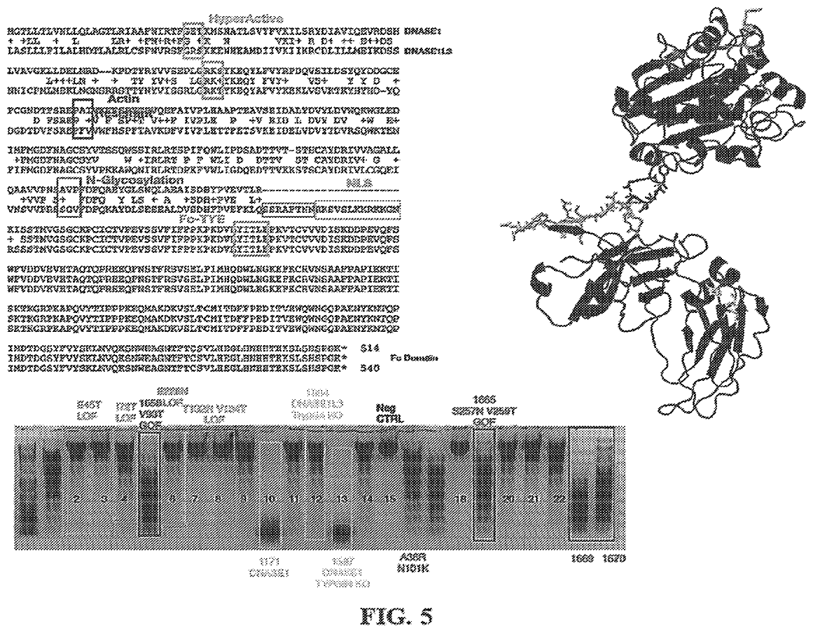

The sequence alignment of mouse DNAse1 (SEQ ID NO:42, ‘query’ below) and mouse DNAse1L3 (SEQ ID NO:43, ‘Sbjct’ below), as shown in FIG. 5 herein, follows:

| | | | |
|---|---|---|---|
| Query | 7 | MGTLLTLVNLLQLAGTLRIAAFNIRTFGETKMSNATLSVYFVKILSRYDIAVIQEVRDSH | 66 |
| | | + +LL + LL R+ +FN+R+FG +K N VKI+R D+ ++ E++DS | |
| Sbjct | 10 | LASLLLFILALHDTLALRLCSFNVRSFGRSKKENHEAMDIIVKIIKRCDLILLMEIKDSS | 69 |
| Query | 67 | LVRVGKLLDELNRD--KPDTYRYVVSEPLGRKSYKSQYLFVYRPDQVSILDSYQYDDGCE | 124 |
| | | L+++LN + + TY YV+S LGRK+YKEQY FVY+ VS+ Y YD + | |
| Sbjct | 70 | NNICPMLMEKLNGNSRRSTTYNYVISSRLGRKTYKEQYAFVYKEKLVSVKTKYHYHD-YQ | 128 |
| Query | 125 | PCGNDTFSREPAIVKFFSPYTEVQEFAIVPLHAAPTEAVSEIDALYDVYLDVWQKWGLED | 184 |
| | | D FSREP +V F SP+T V++F IVPLH P +V EIDLDVYDV +W E+ | |
| SbjCt | 129 | DGDTDVFSREPFVVWFHSPFTAVKDFVIVPLHTTPETSVKEIDELVDVYTDVRSQWKTEN | 188 |
| Query | 185 | IMFMGDFNAGCSYVTSSQWSSIRLRTSPIFQWLIPDSADTTVT-STHCAYDRIVVAGALL | 243 |
| | | +FMGDFNAGCSYV W +IRLRT P F WLI D DTTV ST CAYDRIV+ G + | |
| Sbjct | 189 | FIFMGDFNAGCSYVPKKAWQNIRLRTDPKFVWLIGDQEDTTVKKSTSCAYDRIVLCGQEI | 248 |
| Query | 244 | QAAVVPNSAVPFDFQAEYGLSNQLAEAISDHYPVEVTLR | 282 |
| | | +VVP S+ FDFQ Y LS + A +SDH+PVE L+ | |
| Sbjct | 249 | VNSVVPRSSGVFDFQKAYDLSEEEALDVSDHFPVEFKLQSSRAFTNNRKSVSLKKRKKGN | 308 |
| Query | 283 | KISSTMVGSGCKPCICTVPEVSSVFIFPPKPKDVLYITLEPKVTCVVVDISKDDPEVQFS | 342 |
| | | + SSTMVGSGCKPCICTVPEVSSVFIFPPKPKDVLYITLEPKVTCVVVDISKDDPEVQFS | |
| Sbjct | 309 | RSSSTMVGSGCKPCICTVPEVSSVFIFPPKPKDVLYITLEPKVTCVVVDISKDDPEVQFS | 368 |
| Query | 343 | WFVDDVEVHTAQTQPREEQFNSTFRSVSELPIMHQDWLNGKEFKCRVNSAAFPAPIEKTI | 402 |
| | | WFVDDVEVHTAQTQPREEQFNSTFRSVSELPIMHQDWLNGKEFKCRVNSAAFPAPIEKTI | |
| Sbjct | 369 | WFVDDVEVHTAQTQPREEQFNSTFRSVSELPIMHQDWLNGKEFKCRVNSAAFPAPIEKTI | 428 |
| Query | 403 | SKIRGRPKAFQVYTIPPPKEQMAKDKVSLTCMITDEEPEDLTVEWQWNGQPAENYKNTQP | 462 |
| | | SKTKGRPKAPQVYTIPPPKEQMAKDKVSLTCMITDFFPEDITVEWQWNGQPAENYKNTQP | |
| Sbjct | 429 | SKTKGRPKAPQVYTIPPPKEQMAKDKVSLTCMITDFFPEDITVEWQWNGQPAENYKNTQP | 488 |
| Query | 463 | IMDTDGSYFVYSKLNVQKSNWEAGNTFTCSVLHEGLHNHHTEKSLSHSPGK* | 514 |
| | | IMDTDGSYFVYSKLNVQKSNWEAGNTFTCSVLHEGLHNHHTEKSLSHSPGK* | |
| Sbjct | 489 | IMDTDGSYFVYSKLNVOKSNWEAGNTFTCSVLHEGLHNHHTEKSLSHSPGK* | 540 |

As used herein, the term “Fc” refers to a human IgG (immunoglobulin) Fc domain. Subtypes of IgG such as IgG1, IgG2, IgG3, and IgG4 are contemplated for usage as Fc domains.

As used herein, the “Fc region” is the portion of an IgG molecule that correlates to a crystallizable fragment obtained by papain digestion of an IgG molecule. The Fc region comprises the C-terminal half of the two heavy chains of an IgG molecule that are linked by disulfide bonds. It has no antigen binding activity but contains the carbohydrate moiety and the binding sites for complement and Fc receptors, including the FcRn receptor. The Fc fragment contains the entire second constant domain CH2 (residues 231-340 of human IgG1, according to the Kabat numbering system) and the third constant domain CH3 (residues 341-447). The term “IgG hinge-Fc region” or “hinge-Fc fragment” refers to a region of an IgG molecule consisting of the Fc region (residues 231-447) and a hinge region (residues 216-230) extending from the N-terminus of the Fc region. The term “constant domain” refers to the portion of an immunoglobulin molecule having a more conserved amino acid sequence relative to the other portion of the immunoglobulin, the variable domain, which contains the antigen binding site. The constant domain contains the CH1, CH2 and CH3 domains of the heavy chain and the CHL domain of the light chain.

As used herein, the term “Fc receptors” refer to proteins found on the surface of certain cells (including, among others, B lymphocytes, follicular dendritic cells, natural killer cells, macrophages, neutrophils, eosinophils, basophils, human platelets, and mast cells) that contribute to the protective functions of the immune system. Fc receptors bind to antibodies that are attached to infected cells or invading pathogens. Immunoglobulin Fc receptors (FcRs) are expressed on all hematopoietic cells and play crucial roles in antibody-mediated immune responses. Binding of immune complexes to FcR activates effector cells, leading to phagocytosis, endocytosis of IgG-opsonized particles, releases of inflammatory mediators, and antibody-dependent cellular cytotoxicity (ADCC). Fc receptors have been described for all classes of immunoglobulins: FcγR and neonatal FcR (FcRn) for IgG, FcεR for IgE, FcαR for IgA, FcδR for IgD and FcμR for IgM. All known Fc receptors structurally belong to the immunoglobulin superfamily, except for FcRn and FcεRII, which are structurally related to class I Major Histocompatibility antigens and C-type lectins, respectively (Fc Receptors, Neil A. Fangera, et al., in Encyclopedia of Immunology (2^{nd} Edition), 1998).

As used herein, the term “FcRn Receptor” refers to the neonatal Fc receptor (FcRn), also known as the Brambell receptor, which is a protein that in humans is encoded by the FCGRT gene. An FcRn specifically binds the Fc domain of an antibody. FcRn extends the half-life of IgG and serum albumin by reducing lysosomal degradation in endothelial cells. IgG, serum albumin, and other serum proteins are continuously internalized through pinocytosis. Generally, serum proteins are transported from the endosomes to the lysosome, where they are degraded. FcRn-mediated transcytosis of IgG across epithelial cells is possible because FcRn binds IgG at acidic pH (<6.5) but not at neutral or higher pH. IgG and serum albumin are bound by FcRn at the slightly acidic pH (<6.5), and recycled to the cell surface where they are released at the neutral pH (>7.0) of blood. In this way IgG and serum albumin avoid lysosomal degradation.

The Fc portion of an IgG molecule is located in the constant region of the heavy chain, notably in the CH2 domain. The Fc region binds to an Fc receptor (FcRn), which is a surface receptor of a B cell and also proteins of the complement system. The binding of the Fc region of an IgG molecule to an FcRn activates the cell bearing the receptor and thus activates the immune system. The Fc residues critical to the mouse Fc-mouse FcRn and human Fc-human FcRn interactions have been identified (Dall'Acqua et al., 2002, J. Immunol. 169(9):5171-80). An FcRn binding domain comprises the CH2 domain (or a FcRn binding portion thereof) of an IgG molecule.

As used herein, the term “fragment,” as applied to a nucleic acid, refers to a subsequence of a larger nucleic acid. A “fragment” of a nucleic acid can be at least about 15, 50-100, 100-500, 500-1000, 1000-1500 nucleotides, 1500-2500, or 2500 nucleotides (and any integer value in between). As used herein, the term “fragment,” as applied to a protein or peptide, refers to a subsequence of a larger protein or peptide, and can be at least about 20, 50, 100, 200, 300 or 400 amino acids in length (and any integer value in between).

The term “functional equivalent” or “functional derivative” denotes, in the context of a functional derivative of an amino acid sequence, a molecule that retains a biological activity (either function or structural) that is substantially similar to that of sequences of DNAse1-Fc and/or DNAse1E3-Fc constructs shown herein. A functional derivative or equivalent may be a natural derivative or is prepared synthetically. The functionally-equivalent polypeptides of the disclosure can also be polypeptides identified using one or more techniques of structural and or sequence alignment known in art.

Exemplary functional derivatives include amino acid sequences having substitutions, deletions, or additions of one or more amino acids, provided that the biological activity of the protein is conserved. The substituting amino acid desirably has chemico-physical properties which are similar to that of the substituted amino acid. Desirable similar chemico-physical properties include, similarities in charge, bulkiness, hydrophobicity, hydrophilicity, and the like. Typically, greater than 30% identity between two polypeptides is considered to be an indication of functional equivalence. Preferably, functionally equivalent polypeptides of the disclosure have a degree of sequence identity with the DNAse1-Fc and/or DNAse1L3-Fc constructs of greater than 80%. More preferred polypeptides have degrees of identity of greater than 85%, 90%, 95%, 98% or 99%, respectively. Method for determining whether a functional equivalent or functional derivative has the same or similar or higher biological activity than the DNAse1-Fc and/or DNAse1L3-Fc construct can be determined by using enzymology assays known in the art.

“Gene transfer” and “gene delivery” refer to methods or systems for reliably inserting a particular nucleic acid sequence into targeted cells.

An “inducible” promoter is a nucleotide sequence that, when operably linked with a polynucleotide that encodes or specifies a gene product, causes the gene product to be produced in a cell substantially only when an inducer that corresponds to the promoter is present in the cell.

As used herein, the term “in vivo half-life” for a protein and/or polypeptide contemplated within the disclosure (such as, for example, a DNAse1 and/or DNAse1L3 construct containing FcRn binding sites) refers to the time required for half the quantity administered in the animal to be cleared from the circulation and/or other tissues in the animal. When a clearance curve of a fusion protein is constructed as a function of time, the curve is usually biphasic with a rapid a-phase (which represents an equilibration of the administered molecules between the intra- and extra-vascular space and which is, in part, determined by the size of molecules), and a longer β-phase (which represents the catabolism of the molecules in the intravascular space). In certain embodiments, the term “in vivo half-life” in practice corresponds to the half-life of the molecules in the β-phase.

“Instructional material,” as that term is used herein, includes a publication, a recording, a diagram, or any other medium of expression that can be used to communicate the usefulness of the nucleic acid, peptide, and/or compound of the disclosure in the kit for identifying or alleviating or treating the various diseases or disorders recited herein.

“Isolated” means altered or removed from the natural state. For example, a nucleic acid or a polypeptide naturally present in a living animal is not “isolated,” but the same nucleic acid or polypeptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences that are normally adjacent to the fragment, i.e., the sequences adjacent to the fragment in a genome in which it naturally occurs. The term also applies to nucleic acids that have been substantially purified from other components which naturally accompany the nucleic acid, i.e., RNA or DNA or proteins, which naturally accompany it in the cell. The term therefore includes, for example, a recombinant DNA that is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA that is part of a hybrid gene encoding additional polypeptide sequence.

An “oligonucleotide” or “polynucleotide” is a nucleic acid ranging from at least 2, in certain embodiments at least 8, 15 or 25 nucleotides in length, but may be up to 50, 100, 1000, or 5000 nucleotides long or a compound that specifically hybridizes to a polynucleotide.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

As used herein, the term “patient,” “individual” or “subject” refers to a human.

As used herein, the term “pharmaceutical composition” or “composition” refers to a mixture of at least one compound useful within the disclosure with a pharmaceutically acceptable carrier. The pharmaceutical composition facilitates administration of the compound to a patient. Multiple techniques of administering a compound exist in the art including, but not limited to, subcutaneous, intravenous, oral, aerosol, inhalational, rectal, vaginal, transdermal, intranasal, buccal, sublingual, parenteral, intrathecal, intragastrical, ophthalmic, pulmonary, and topical administration.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the patient. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof.

As used herein, the term “polypeptide” refers to a polymer composed of amino acid residues, related naturally occurring structural variants, and synthetic non-naturally occurring analogues thereof linked via peptide bonds.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements that are required for expression of the gene product. The promoter/regulatory sequence may for example be one that expresses the gene product in a tissue specific manner.

The term “recombinant polypeptide” as used herein is defined as a polypeptide produced by using recombinant DNA methods.

The term “recombinant DNA” as used herein is defined as DNA produced by joining pieces of DNA from different sources.

“Sample” or “biological sample” as used herein means a biological material isolated from a subject. The biological sample may contain any biological material suitable for detecting a mRNA, polypeptide or other marker of a physiologic or pathologic process in a subject, and may comprise fluid, tissue, cellular and/or non-cellular material obtained from the individual.

As used herein, the term “signal peptide” refers to a sequence of amino acid residues (ranging in length from, for example, 10-30 residues) bound at the amino terminus of a nascent protein of interest during protein translation. The signal peptide is recognized by the signal recognition particle (SRP) and cleaved by the signal peptidase following transport at the endoplasmic reticulum. (Lodish, et al., 2000, Molecular Cell Biology, 4^{th} edition).

As used herein, “substantially purified” refers to being essentially free of other components. For example, a substantially purified polypeptide is a polypeptide that has been separated from other components with which it is normally associated in its naturally occurring state. Non-limiting embodiments include 95% purity, 99% purity, 99.5% purity, 99.9% purity and 100% purity.

A “tissue-specific” promoter is a nucleotide sequence that, when operably linked with a polynucleotide encodes or specified by a gene, causes the gene product to be produced in a cell substantially only if the cell is a cell of the tissue type corresponding to the promoter.

The phrase “under transcriptional control” or “operatively linked” as used herein means that the promoter is in the correct location and orientation in relation to a polynucleotide to control the initiation of transcription by RNA polymerase and expression of the polynucleotide.

The term “transfected” or “transformed” or “transduced” as used herein refers to a process by which exogenous nucleic acid is transferred or introduced into the host cell. A “transfected” or “transformed” or “transduced” cell has been transfected, transformed or transduced with exogenous nucleic acid. The cell includes the primary subject cell and its progeny.

As used herein, the term “treatment” or “treating” is defined as the application or administration of a therapeutic agent, i.e., a compound useful within the disclosure (alone or in combination with another pharmaceutical agent), to a patient, or application or administration of a therapeutic agent to an isolated tissue or cell line from a patient (e.g., for diagnosis or ex vivo applications), who has a disease or disorder, or a symptom of a disease or disorder, with the purpose to cure, heal, alleviate, relieve, alter, remedy, ameliorate, improve or affect the disease or disorder, or the symptoms of the disease or disorder. Such treatments may be specifically tailored or modified, based on knowledge obtained from the field of pharmacogenomics.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide may differ in amino acid sequence by one or more substitutions, additions, or deletions in any combination. A variant of a nucleic acid or peptide may be a naturally occurring such as an allelic variant, or may be a variant that is not known to occur naturally. Non-naturally occurring variants of nucleic acids and peptides may be made by mutagenesis techniques or by direct synthesis.

A “vector” is a composition of matter that comprises an isolated nucleic acid and that may be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

As used herein, the term “virus” is defined as a particle consisting of nucleic acid (RNA or DNA) enclosed in a protein coat, with or without an outer lipid envelope, which is capable of transfecting the cell with its nucleic acid.

As used herein, the term “wild-type” refers to a gene or gene product isolated from a naturally occurring source. A wild-type gene is most frequently observed in a population and is thus arbitrarily designed the “normal” or “wild-type” form of the gene. In contrast, the term “modified” or “mutant” refers to a gene or gene product that displays modifications in sequence and/or functional properties (i.e., altered characteristics) when compared to the wild-type gene or gene product. Naturally occurring mutants can be isolated; these are identified by the fact that they have altered characteristics (including altered nucleic acid sequences) when compared to the wild-type gene or gene product.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Constructs and Polypeptides

In one aspect, the disclosure provides a DNAse1-Fc and/or DNAse1L3-Fc construct. The disclosure contemplates that the constructs contemplated herein can have one or more of the mutations described herein.

Further, the disclosure provides homodimeric constructs comprising two independently selected DNAse1 constructs of the disclosure. Further, the disclosure provides homodimeric constructs comprising two independently selected DNAse1L3 constructs of the disclosure. Further, the disclosure provides heterodimeric constructs comprising a DNAse1 construct of the disclosure and a DNAse1L3 construct of the disclosure.

The disclosure provides the constructs described herein, as well as any glycosylation variants (alternative glycoforms), as well as constructs that have been modified through site-directed mutagenesis or any sort of protein chemistry manipulation so as to have improved solubility and/or enzymatic activity and/or in vivo half-life.

In certain embodiments, the construct comprises the amino acid sequence:

DNAse1-X1-LINKER-Fc-X2 (I)

wherein:
DNAse1 is a human DNAse1 polypeptide as described elsewhere herein;
X1 is a covalent bond, or X1 is the peptide of amino acid sequence RAFTNNRKSVSLKKRKKGNRS (SEQ ID NO:3) or a fragment thereof;
LINKER is a chemical bond or a polypeptide comprising 1-100 amino acids;
X2 is null, or X2 is the peptide of amino acid sequence RAFTNNRKSVSLKKRKKGNRS (SEQ ID NO:3) or a fragment thereof;
Fc is the Fc domain of human IgG1 as described elsewhere herein.

In certain embodiments, (I) describes the construct from left to right as from its N-terminus to its C-terminus. In that case, the N-terminus of the Fc is linked to the C-terminus of the DNAse1. In certain embodiments, (I) describes the construct from left to right as from its C-terminus to its N-terminus. In that case, the C-terminus of the Fc is linked to the N-terminus of the DNAse1.

In certain embodiments, the polypeptide comprises the amino acid sequence:

DNAse1L3-X1-LINKER-Fc-X2 (II)

wherein:
DNAse1L3 is a human polypeptide DNAse1L3 as described elsewhere herein;
X1 is a covalent bond, or X1 is the peptide of amino acid sequence RAFTNNRKSVSLKKRKKGNRS (SEQ ID NO:3) or a fragment thereof;
LINKER is a covalent bond or a polypeptide comprising 1-100 amino acids;
X2 is null, or X2 is the peptide of amino acid sequence RAFTNNRKSVSLKKRKKGNRS (SEQ ID NO:3) or a fragment thereof;
Fc is the Fc domain of human IgG1 as described elsewhere herein.

In certain embodiments, (II) describes the construct from left to right as from its N-terminus to its C-terminus. In that case, the N-terminus of the Fc is linked to the C-terminus of the DNAse1L3. In certain embodiments, (II) describes the construct from left to right as from its C-terminus to its N-terminus. In that case, the C-terminus of the Fc is linked to the N-terminus of the DNAse1L3.

### Fc:

In certain embodiments, the Fc domain of human IgG1 has the following sequence:

hIgG Fc domain, Fc(human)
SEQ ID NO: 4
DKTHTCPPCPAPELLGGPSVFLFPPKPKDTL
MISRTPEVTCVVVDVSHEDPEVKFNWYVDGV
EVHNAKTKPREEQYNSTYRVVSVLTVLHQDW
LNGKEYKCKVSNKALPAPIEKTISKAKGQPR
EPQVYTLPPSREEMTKNQVSLTCLVKGFYPS
DIAVEWESNGQPENNYKTTPPVLDSDGSFFL
YSKLTVDKSRWQQGNVFSCSVMHEALHNHYT
QKSLSLSPGK

In certain embodiments, the Fc domain of mouse IgG1 has the following sequence:

hIgG Fc domain, Fc(mouse)
SEQ ID NO: 31
GCKPCICTVPEVSSVFIFPPKPKDVLYITLE
PKVTCVVVDISKDDPEVQFSWFVDDVEVHTA
QTQPREEQFNSTFRSVSELPIMHQDWLNGKE
FKCRVNSAAFPAPIEKTISKTKGRPKAPQVY
TIPPPKEQMAKDKVSLTCMITDFFPEDITVE
WQWNGQPAENYKNTQPIMDTDGSYFVYSKLN
VQKSNWEAGNTFTCSVLHEGLHNHHTEKSLS
HSPGK

In certain embodiments, Cys6 (C6) with respect to SEQ ID NO:4 is mutated to another amino acid, such as but not limited to G or S. In certain embodiments, Cys9 (C9) with respect to SEQ ID NO:4 is mutated to another amino acid, such as but not limited to Gly or Ser. In a non-limiting embodiment, any one of such mutations in the C6/C9 residues responsible for the interchain disulfide bond in the heavy chain of the Fc domain converts a dimeric enzyme fusion to a monomeric fusion, thus allowing for greater accessibility to chromatin and microparticle DNA.

In certain embodiments, the hIgG Fc domain has at least one of the following mutations with respect to SEQ ID NO:4: M32Y, S34T, and T36E. In a non-limiting embodiment, any such mutations enhances endosomal recycling of the corresponding construct. In certain embodiments, the hIgG Fc domain has the following mutations with respect to SEQ ID NO:4: M32Y, S34T, and T36E.

A non-limiting list of contemplated mutations in the Fc domain of the constructs of the disclosure include C6S, C9S, M32Y, S34T, and/or T36E with respect to SEQ ID NO:4. In certain embodiments, the Fc domain of the construct comprise the C6S mutation with respect to SEQ ID NO:4. In certain embodiments, the Fc domain of the construct comprise the C9S mutation with respect to SEQ ID NO:4. In certain embodiments, the Fc domain of the construct comprise the M32Y mutation with respect to SEQ ID NO:4. In certain embodiments, the Fc domain of the construct comprise the S34T mutation with respect to SEQ ID NO:4. In certain embodiments, the Fc domain of the construct comprise the T36E mutation with respect to SEQ ID NO:4.

### LINKER:

In certain embodiments, the LINKER is a chemical bond or absent. In certain embodiments, the LINKER is a polypeptide comprising 1-100, 1-90, 1-80, 1-70, 1-60, 1-50, 1-40, 1-30, 1-20, 1-10, and/or 1-5 amino acids. In certain embodiments, the LINKER comprises Gly and/or Ser amino acids.

In certain embodiments, the LINKER comprises GS.

In certain embodiments, the LINKER comprises GSC.

In certain embodiments, the LINKER comprises GGGGSGGGGS (SEQ ID NO:5).

In certain embodiments, the LINKER comprises SSTMVRS (SEQ ID NO:40).

In certain embodiments, the LINKER comprises SSTMVGS (SEQ ID NO:41).

In certain embodiments, the LINKER comprises ELKTPLGDTTHTXPRZPAPELLGGP (SEQ ID NO:6), wherein each occurrence of X is C, G, or S, and wherein each occurrence of Z is C, G, or S. In certain non-limiting embodiments, at least one of X and Z is not C and formation of a disulfide bridge is prevented. In certain embodiments, SEQ ID NO:6 corresponds to the hinge region of Human IgG1.

### X1 and X2:

In certain embodiments, X1 is a covalent bond. In certain embodiments, X1 is the peptide of amino acid sequence RAFTNNRKSVSLKKRKKGNRS (SEQ ID NO:3) or a fragment thereof.

In certain embodiments, X2 is a covalent bond. In certain embodiments, X2 is the peptide of amino acid sequence RAFTNNRKSVSLKKRKKGNRS (SEQ ID NO:3) or a fragment thereof.

### DNAse1:

An illustrative construct of the disclosure comprises the amino acid sequence of SEQ ID NO:7, wherein the bold sequence corresponds to the DNAse1 polypeptide, wherein the underlined sequence corresponds to the Fc, and wherein the italics sequence corresponds to the LINKER.

TFGETKMSNATLVSYIVQILSRYDIALVQEV
RDSHLTAVGKLLDNLNQDAPDTYHYVVSEPL
GRNSYKERYLFVYRPDQVSAVDSYYYDDGCE
PCGNDTFNREPAIVRFFSRFTEVREFAIVPL
HAAPGDAVAEIDALYDVYLDVQEKWGLEDVM
LMGDFNAGCSYVRPSQWSSIRLWTSPTFQWL
IPDSADTTATPTHCAYDRIVVAGMLLRGAVV
PDSALPFNFQAAYGLSDQLAQAISDHYPVEV
MLKGSDKTHTCPPCPAPELLGGPSVFLFPPK
PKDTLMISRTPEVTCVVVDVSHEDPEVKFNW
YVDGVEVHNAKTKPREEQYNSTYRVVSVLTV
LHQDWLNGKEYKCKVSNKALPAPIEKTISKA
KGQPREPQVYTLPPSREEMTKNQVSLTCLVK
GFYPSDIAVEWESNGQPENNYKTTPPVLDSD
GSFFLYSKLTVDKSRWQQGNVFSCSVMHEAL
HNHYTQKSLSLSPGK

In certain embodiments, the construct has one or more of the following mutations in Fc: C290S, C293S, M316Y, S318T, and/or T320E with respect to SEQ ID NO:7.

An illustrative construct of the disclosure comprises the amino acid sequence of SEQ ID NO:8, wherein the bold sequence corresponds to the DNAse1 polypeptide, wherein the underlined sequence corresponds to the Fc, and wherein the italics sequence corresponds to the LINKER.

TFGETKMSNATLVSYIVQILSRYDIALVQEV
RDSHLTAVGKLLDNLNQDAPDTYHYVVSEPL
GRNSYKERYLFVYRPDQVSAVDSYYYDDGCE
PCGNDTFNREPAIVRFFSRFTEVREFAIVPL
HAAPGDAVAEIDALYDVYLDVQEKWGLEDVM
LMGDFNAGCSYVRPSQWSSIRLWTSPTFQWL
IPDSADTTATPTHCAYDRIVVAGMLLRGAVV
PDSALPFNFQAAYGLSDQLAQAISDHYPVEV
MLKGSDKTHTSPPSPAPELLGGPSVFLFPPK
PKDTLYITREPEVTCVVVDVSHEDPEVKFNW
YVDGVEVHNAKTKPREEQYNSTYRVVSVLTV
LHQDWLNGKEYKCKVSNKALPAPIEKTISKA
KGQPREPQVYTLPPSREEMTKNQVSLTCLVK
GFYPSDIAVEWESNGQPENNYKTTPPVLDSD
GSFFLYSKLTVDKSRWQQGNVFSCSVMHEAL
HNHYTQKSLSLSPGK

In certain embodiments, the construct lacks at least a portion of the signal peptide of DNAse1 corresponding to residues 1-22 of SEQ ID NO:1. In certain embodiments, the construct lacks the signal peptide of DNAse1 corresponding to residues 1-22 of SEQ ID NO:1.

A non-limiting list of contemplated mutations in the DNAse1 domain of the constructs of the disclosure with respect to SEQ ID NO:1 include but are not limited to Q31R, E35R, Y46H, Y46S, V88N, N96K, D109N, V111T, A136F, R148S, E149N, M186I, L208P, D220N, D250N, A252T, G262N, D265N, and L267T.

In certain embodiments, the DNAse1 domain of the construct comprises the mutation Q31R with respect to SEQ ID NO:1. In certain embodiments, the DNAse1 domain of the construct comprises the mutation E35R with respect to SEQ ID NO:1. In certain embodiments, the DNAse1 domain of the construct comprises the mutation Y46H with respect to SEQ ID NO:1. In certain embodiments, the DNAse1 domain of the construct comprises the mutation Y46S with respect to SEQ ID NO:1. In certain embodiments, the DNAse1 domain of the construct comprises the mutation V88N with respect to SEQ ID NO:1. In certain embodiments, the DNAse1 domain of the construct comprises the mutation N96K with respect to SEQ ID NO:1. In certain embodiments, the DNAse1 domain of the construct comprises the mutation D109N with respect to SEQ ID NO:1. In certain embodiments, the DNAse1 domain of the construct comprises the mutation V111T with respect to SEQ ID NO:1. In certain embodiments, the DNAse1 domain of the construct comprises the mutation A136F with respect to SEQ ID NO:1. In certain embodiments, the DNAse1 domain of the construct comprises the mutation R148S with respect to SEQ ID NO:1. In certain embodiments, the DNAse1 domain of the construct comprises the mutation E149N with respect to SEQ ID NO:1. In certain embodiments, the DNAse1 domain of the construct comprises the mutation M186I with respect to SEQ ID NO:1. In certain embodiments, the DNAse1 domain of the construct comprises the mutation L208P with respect to SEQ ID NO:1. In certain embodiments, the DNAse1 domain of the construct comprises the mutation D220N with respect to SEQ ID NO:1. In certain embodiments, the DNAse1 domain of the construct comprises the mutation D250N with respect to SEQ ID NO:1. In certain embodiments, the DNAse1 domain of the construct comprises the mutation A252T with respect to SEQ ID NO:1. In certain embodiments, the DNAse1 domain of the construct comprises the mutation G262N with respect to SEQ ID NO:1. In certain embodiments, the DNAse1 domain of the construct comprises the mutation D265N with respect to SEQ ID NO:1. In certain embodiments, the DNAse1 domain of the construct comprises the mutation L267T with respect to SEQ ID NO:1.

In certain non-limiting embodiments, the mutation A136F with respect to SEQ ID NO:1 decreases actin binding of the construct.

In certain non-limiting embodiments, the mutation(s) E35R, Y46H, Y46S, R148S, E149N, M186I, L208P, and/or D220N increase the enzymatic activity of the construct.

In certain non-limiting embodiments, the mutation(s) V88N, D109N, V111T, G262N, D265N, and/or L267T modify the overall glycosylation status of the construct.

Non-limiting examples of constructs of the disclosure comprise the following amino acid sequences, wherein the bold sequence corresponds to the DNAse1 polypeptide, wherein the underlined sequence corresponds to the Fc, wherein the italics sequence corresponds to the LINKER, and wherein the italics/underlined sequence corresponds to X1/X2. Certain mutations are shown as doubly underlined.

SEQ ID NO: 9
MRGMKLLGALLALAALLQGAVSLKIAAFNIQTFGRTKMSNATLVSYIVQILSRYDIALVQEV
RDSHLTAVGKLLDNLNQDAPDTYHYNVSEPLGRNSYKERYLFVYRPNQTSAVDSYYYDDGCE
PCGNDTFNREPFIVRFFSRFTEVREFAIVPLHAAPGDAVAEIDALYDVYLDVQEKWGLEDVM
LMGDFNAGCSYVRPSQWSSIRLWTSPTFQWLIPDSADTTATPTHCAYDRIVVAGMLLRGAVV
PDSALPFNFQAAYNLSNQTAQAISDHYPVEVMLKGSDKTHTSPPSPAPELLGGPSVFLFPPK
PKDTLYITREPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSVMHEAL
HNHYTQKSLSLSPGK
SEQ ID NO: 10
MRGMKLLGALLALAALLQGAVSLKIAAFNIQTFGETKMSNATLVSYIVQILSRYDIALVQEV
RDSHLTAVGKLLDNLNQDAPDTYHYVVSEPLGRNSYKERYLFVYRPDQVSAVDSYYYDDGCE
PCGNDTFNREPAIVRFFSRFTEVREFAIVPLHAAPGDAVAEIDALYDVYLDVQEKWGLEDVM
LMGDFNAGCSYVRPSQWSSIRLWTSPTFQWLIPDSADTTATPTHCAYDRIVVAGMLLRGAVV
PDSALPFNFQAAYGLSDQLAQAISDHYPVEVMLKRAFTNNRKSVSLKKRKKGNRSGSDKTHT
SPPSPAPELLGGPSVFLFPPKPKDTLYITREPEVTCVVVDVSHEDPEVKFNWYVDGVEVHNA
KTKPREEQYNSTYRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGKRAFTNNRKSVSLKKRKKGNRS
SEQ ID NO: 11
MRGMKLLGALLALAALLQGAVSLKIAAFNIQTFGETKMSNATLVSYIVQILSRYDIALVQEV
RDSHLTAVGKLLDNLNQDAPDTYHYVVSEPLGRNSYKERYLFVYRPDQVSAVDSYYYDDGCE
PCGNDTFNREPAIVRFFSRFTEVREFAIVPLHAAPGDAVAEIDALYDVYLDVQEKWGLEDVM
LMGDFNAGCSYVRPSQWSSIRLWTSPTFQWLIPDSADTTATPTHCAYDRIVVAGMLLRGAVV
PDSALPFNFQAAYGLSDQLAQAISDHYPVEVMLKGSDKTHTSPPSPAPELLGGPSVFLFPPK
PKDTLYITREPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSVMHEAL
HNHYTQKSLSLSPGKRAFTNNRKSVSLKKRKKGNRS
SEQ ID NO: 12
MRGMKLLGALLALAALLQGAVSLKIAAFNIQTFGETKMSNATLVSYIVQILSRYDIALVQEV
RDSHLTAVGKLLDNLNQDAPDTYHYVVSEPLGRNSYKERYLFVYRPDQVSAVDSYYYDDGCE
PCGNDTFNREPAIVRFFSRFTEVREFAIVPLHAAPGDAVAEIDALYDVYLDVQEKWGLEDVM
LMGDFNAGCSYVRPSQWSSIRLWTSPTFQWLIPDSADTTATPTHCAYDRIVVAGMLLRGAVV
PDSALPFNFQAAYGLSDQLAQAISDHYPVEVMLKRAFTNNRKSVSLKKRKKGNRSGSDKTHT
SPPSPAPELLGGPSVFLFPPKPKDTLYITREPEVTCVVVDVSHEDPEVKFNWYVDGVEVHNA
KTKPREEQYNSTYRVVSVLTVLHQDWLNGKEYKCKVSNKALPAPIEKTISKAKGQPREPOVY
TLPPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGK
SEQ ID NO: 13
MRGMKLLGALLALAALLQGAVSLKIAAFNIQTFGETKMSNATLVSYIVQILSRYDIALVQEV
RDSHLTAVGKLLDNLNQDAPDTYHYVVSEPLGRNSYKERYLFVYRPDQVSAVDSYYYDDGCE
PCGNDTFNREPAIVRFFSRFTEVREFAIVPLHAAPGDAVAEIDALYDVYLDVQEKWGLEDVM
LMGDFNAGCSYVRPSQWSSIRLWTSPTFQWLIPDSADTTATPTHCAYDRIVVAGMLLRGAVV
PDSALPFNFQAAYGLSDQLAQAISDHYPVEVMLKGGGGSGGGGSDKTHTSPPSPAPELLGGP
SVFLFPPKPKDTLYITREPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTVDKSRWQQGNVFS
CSVMHEALHNHYTQKSLSLSPGK
SEQ ID NO: 14
MRGMKLLGALLALAALLQGAVSLKIAAFNIQTFGETKMSNATLVSYIVQILSRYDIALVQEV
RDSHLTAVGKLLDNLNQDAPDTYHYVVSEPLGRNSYKERYLFVYRPDQVSAVDSYYYDDGCE
PCGNDTFNREPAIVRFFSRFTEVREFAIVPLHAAPGDAVAEIDALYDVYLDVQEKWGLEDVM
LMGDFNAGCSYVRPSQWSSIRLWTSPTFQWLIPDSADTTATPTHCAYDRIVVAGMLLRGAVV
PDSALPFNFQAAYGLSDQLAQAISDHYPVEVMLKELKTPLGDTTHTXPRZPAPELLGGPDKT
HTSPPSPAPELLGGPSVFLFPPKPKDTLYITREPEVTCVVVDVSHEDPEVKFNWYVDGVEVH
NAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSK
LTVDKSRWQQGNVFSCSVMHEALHNHYTQKSLSLSPGK

wherein X and Z are independently Cys, Gly, or Ser.

In certain non-limiting embodiments, wherein at least one of X and Z is not Cys (C) formation of disulfide bridge is prevented.

SEQ ID NO: 15
MRGMKLLGALLALAALLQGAVSLKIAAFNI [symbol] TFGETKMSNATLVSYIVQILSRYDIALVQEV
RDSHLTAVGKLLDNLNQDAPDTYHYVVSEPLGR [symbol] SYKERYLFVYRPDQVSAVDSYYYDDGCE
PCGNDTFNREP [symbol] IVRFFSRFTEVREFAIVPLHAAPGDAVAEIDALYDVYLDVQEKWGLEDVM
LMGDFNAGCSYVRPSQWSSIRLWTSPTFQWLIPDSADTTATPTHCAYDRIVVAGMLLQGAVV
PDSALPFNFQAAYGLSDQLAQAISDHYPVEVMLKGSCDKTHTCPPCPAPELLGGPSVFLFPP
KPKDTLYITREPEVTCVVVDVSHEDPEVKENWYVDGVEVHNAKTKPREEQYNSTYRVVSVLT
VLHQDWLNGKEYKCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQGNVFSCSVMHEA
LHNHYTOKSLSLSPGK
SEQ ID NO: 16
MRGMKLLGALLALAALLQGAVSLKIAAFNI [symbol] TFGETKMSNATLVSYIVQILSRYDIALVQEV
RDSHLTAVGKLLDNLNQDAPDTYHYVVSEPLGR [symbol] SYKERYLFVYRPDQVSAVDSYYYDDGCE
PCGNDTFNREP [symbol] IVRFFSRFTEVREFAIVPLHAAPGDAVAEIDALYDVYLDVQEKWGLEDVM
LMGDFNAGCSYVRPSQWSSIRLWTSPTFQWLIPDSADTTATPTHCAYDRIVVAGMLLQGAVV
PDSALPFNFQAAYGLS [symbol] Q [symbol] AQAISDHYPVEVMLKGSCDKTHTCPPCPAPELLGGPSVFLFPP
KPKDTLYITREPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQGNVFSCSVMHEA
LHNHYTQKSLSLSPGK
SEQ ID NO: 17
MRGMKLLGALLALAALLQGAVSLKIAAFNI [symbol] TFGETKMSNATLVSYIVQILSRYDIALVQEV
RDSHLTAVGKLLDNLNQDAPDTYHYVVSEPLGR [symbol] SYKERYLFVYRPDQVSAVDSYYYDDGCE
PCGNDTFNREP [symbol] IVRFFSRFTEVREFAIVPLHAAPGDAVAEIDALYDVYLDVQEKWGLEDVM
LMGDFNAGCSYVRPSQWSSIRLWTSPTFQWLIPDSADTTATPTHCAYDRIVVAGMLLQGAVV
P [symbol] S [symbol] LPFNFQAAYGLSDQLAQAISDHYPVEVMLKGSCDKTHTCPPCPAPELLGGPSVFLFPP
KPKDTLYITREPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQGNVFSCSVMHEA
LHNHYTQKSLSLSPGK

### DNAse1L3:

An illustrative construct of the disclosure comprises the amino acid sequence of SEQ ID NO:18, wherein the bold sequence corresponds to the DNAse1L3 polypeptide, wherein the underlined sequence corresponds to the Fc, and wherein the italics sequence corresponds to the LINKER.

SEQ ID NO: 18
MSRELAPLLLLLLSIHSALAMRICSFNVRSF
GESKQEDKNAMDVIVKVIKRCDIILVMEIKD
SNNRICPILMEKLNRNSRRGITYNYVISSRL
GRKTYKEQYAFLYKEKLVSVKRSYHYHDYQD
GDADVFSREPFVVWFQSPHTAVKDFVIIPLH
TTPETSVKEIDELVEVYTDVKHRWKAENFIF
MGDFNAGCSYVPKKAWKNIRLRTDPRFVWLI
GDQEDTTVKKSTNCAYDRIVLRGQEIVSSVV
PKSNSVFDFQKAYKLTEEEALDVSDHFPVEF
KLQSSRAFTNSKKSVTLRKKTKSKRSGSDKT
HTCPPCPAPELLGGPSVFLFPPKPKDTLMIS
RTPEVTCVVVDVSHEDPEVKFNWYVDGVEVH
NAKTKPREEQYNSTYRVVSVLTVLHQDWLNG
KEYKCKVSNKALPAPIEKTISKAKGQPREPQ
VYTLPPSREEMTKNQVSLTCLVKGFYPSDIA
VEWESNGQPENNYKTTPPVLDSDGSFFLYSK
LTVDKSRWQQGNVFSCSVMHEALHNHYTQKS
LSLSPGK

In certain embodiments, the construct has one or more of the following mutations in Fc: C313S, C316S, M339Y, S341T, and/or T342E with respect to SEQ ID NO:18.

An illustrative construct of the disclosure comprises the amino acid sequence of SEQ ID NO:19, wherein the bold sequence corresponds to the DNAse1L3 polypeptide, wherein the underlined sequence corresponds to the Fc, and wherein the italics sequence corresponds to the LINKER.

SEQ ID NO: 19
MSRELAPLLLLLLSIHSALAMRICSFNVRSF
GESKQEDKNAMDVIVKVIKRCDIILVMEIKD
SNNRICPILMEKLNRNSRRGITYNYVISSRL
GRKTYKEQYAFLYKEKLVSVKRSYHYHDYQD
GDADVFSREPFVVWFQSPHTAVKDFVIIPLH
TTPETSVKEIDELVEVYTDVKHRWKAENFIF
MGDFNAGCSYVPKKAWKNIRLRTDPRFVWLI
GDQEDTTVKKSTNCAYDRIVLRGQEIVSSVV
PKSNSVFDFQKAYKLTEEEALDVSDHFPVEF
KLQSSRAFTNSKKSVTLRKKTKSKRSGSDKT
HTSPPSPAPELLGGPSVFLFPPKPKDTLYIT
REPEVTCVVVDVSHEDPEVKFNWYVDGVEVH
NAKTKPREEQYNSTYRVVSVLTVLHQDWLNG
KEYKCKVSNKALPAPIEKTISKAKGQPREPQ
VYTLPPSREEMTKNQVSLTCLVKGFYPSDIA
VEWESNGQPENNYKTTPPVLDSDGSFFLYSK
LTVDKSRWQQGNVFSCSVMHEALHNHYTQKS
LSLSPGK

In certain embodiments, the construct lacks at least a portion of the signal peptide of DNAse1L3 corresponding to residues 1-20 of SEQ ID NO:2. In certain embodiments, the construct lacks the signal peptide of DNAse1L3 corresponding to residues 1-20 of SEQ ID NO:2.

In certain embodiments, the construct lacks at least a portion of the nuclear localization sequence (NLS) of the DNAse1L3 polypeptide. In certain embodiments, the construct lacks residues 291-305 of SEQ ID NO:2. In certain embodiments, the construct lacks residues 292-304 of SEQ ID NO:2. In certain embodiments, the construct lacks residues 296-304 of SEQ ID NO:2. In certain embodiments, the construct lacks residues A-B of SEQ ID NO:2, wherein A ranges from 291 to 296 and B ranges from 304 to 305.

A non-limiting list of contemplated mutations in the Fc domain of the constructs of the disclosure, with respect to SEQ ID NO:18, include C313S, C316S, M339Y, S341T, and/or T342E.

A non-limiting list of contemplated mutations in the DNAse1L3 domain of the constructs of the disclosure, with respect to SEQ ID NO:2, include E33R, M42T, V44H, V88T, N96K, A127N, V129T, K147S, D148N, L207P, D219N, and/or V254T.

In certain embodiments, the DNAseIL3 domain of the construct comprises the mutation E33R with respect to SEQ ID NO:2. In certain embodiments, the DNAseIL3 domain of the construct comprises the mutation M42T with respect to SEQ ID NO:2. In certain embodiments, the DNAseIL3 domain of the construct comprises the mutation V44H with respect to SEQ ID NO:2. In certain embodiments, the DNAseIL3 domain of the construct comprises the mutation V88T with respect to SEQ ID NO:2. In certain embodiments, the DNAseIL3 domain of the construct comprises the mutation N96K with respect to SEQ ID NO:2. In certain embodiments, the DNAseIL3 domain of the construct comprises the mutation A127N with respect to SEQ ID NO:2. In certain embodiments, the DNAseIL3 domain of the construct comprises the mutation V129T with respect to SEQ ID NO:2. In certain embodiments, the DNAseIL3 domain of the construct comprises the mutation K147S with respect to SEQ ID NO:2. In certain embodiments, the DNAseIL3 domain of the construct comprises the mutation D148N with respect to SEQ ID NO:2. In certain embodiments, the DNAseIL3 domain of the construct comprises the mutation L207P with respect to SEQ ID NO:2. In certain embodiments, the DNAseIL3 domain of the construct comprises the mutation D219N with respect to SEQ ID NO:2. In certain embodiments, the DNAseIL3 domain of the construct comprises the mutation V254T with respect to SEQ ID NO:2.

In certain non-limiting embodiments, the mutation A136F with respect to SEQ ID NO:1 decreases actin binding of the construct.

In certain non-limiting embodiments, the mutation(s) E33R, V44H, N96K, K147S, D148N, L207P, and/or D219N with respect to SEQ ID NO:1 increase(s) the enzymatic activity of the construct.

In certain non-limiting embodiments, the mutation V254T modifies the overall glycosylation status of the construct.

Non-limiting examples of constructs of the disclosure comprise the following amino acid sequences, wherein the bold sequence corresponds to the DNAse1L3 polypeptide, wherein the underlined sequence corresponds to the Fc, wherein the italics sequence corresponds to the LINKER, and wherein the italics/underlined sequence corresponds to X1/X2. Certain mutations are shown as doubly underlined.

SEQ ID NO: 20
MSRELAPLLLLLLSIHSALAMRICSFNVRSFGESKQEDKNAMDVIVKVIKRCDIILVMEIKD
SNNRICPILMEKLNRNSRRGITYNYVISSRLGRKTYKEQYAFLYKEKLVSVKRSYHYHDYQD
GDADVFSREPFVVWFQSPHTAVKDFVIIPLHTTPETSVKEIDELVEVYTDVKHRWKAENFIF
MGDFNAGCSYVPKKAWKNIRLRTDPRFVWLIGDQEDTTVKKSTNCAYDRIVLRGQEIVSSVV
PKSNSVFDFQKAYKLTEEEALDVSDHFPVEFKLQSSRAFTNSKKSVTLRKKTKSKRSGSDKT
HTSPPSPAPELLGGPSVFLFPPKPKDTLYITREPEVTCVVVDVSHEDPEVKFNWYVDGVEVH
NAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSK
LTVDKSRWQQGNVFSCSVMHEALHNHYTQKSLSLSPGKRAFTNNRKSVSLKKRKKGNRS
SEQ ID NO: 21
MSRELAPLLLLLLSIHSALAMRICSFNVRSFGESKQEDKNAMDVIVKVIKRCDIILVMEIKD
SNNRICPILMEKLNRNSRRGITYNYVISSRLGRKTYKEQYAFLYKEKLVSVKRSYHYHDYQD
GDADVFSREPFVVWFQSPHTAVKDFVIIPLHTTPETSVKEIDELVEVYTDVKHRWKAENFIF
MGDFNAGCSYVPKKAWKNIRLRTDPRFVWLIGDQEDTTVKKSTNCAYDRIVLRGQEIVSSVV
PKSNSVFDFQKAYKLTEEEALDVSDHFPVEFKLQSSRAFTNSKKSVTLRKKTKSKRSRAFTN
NRKSVSLKKRKKGNRSGSDKTHTSPPSPAPELLGGPSVFLFPPKPKDTLYITREPEVTCVVV
DVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGKRAF
TNNRKSVSLKKRKKGNRS
SEQ ID NO: 22
MSRELAPLLLLLLSIHSALAMRICSFNVRSFGESKQEDKNAMDVIVKVIKRCDIILVMEIKD
SNNRICPILMEKLNRNSRRGITYNYVISSRLGRKTYKEQYAFLYKEKLVSVKRSYHYHDYQD
GDADVFSREPFVVWFQSPHTAVKDFVIIPLHTTPETSVKEIDELVEVYTDVKHRWKAENFIF
MGDFNAGCSYVPKKAWKNIRLRTDPRFVWLIGDQEDTTVKKSTNCAYDRIVLRGQEIVSSVV
PKSNSVFDFQKAYKLTEEEALDVSDHFPVEFKLQSSRAFTNSKKSVTLRKKTKSKRSRAFTN
NRKSVSLKKRKKGNRSGSDKTHTSPPSPAPELLGGPSVFLFPPKPKDTLYITREPEVTCVVV
DVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGK
SEQ ID NO: 23
MSRELAPLLLLLLSIHSALAMRICSFNVRSFGESKQEDKNAMDVIVKVIKRCDIILVMEIKD
SNNRICPILMEKLNRNSRRGITYNYVISSRLGRKTYKEQYAFLYKEKLVSVKRSYHYHDYQD
GDADVFSREPFVVWFQSPHTAVKDFVIIPLHTTPETSVKEIDELVEVYTDVKHRWKAENFIF
MGDFNAGCSYVPKKAWKNIRLRTDPRFVWLIGDQEDTTVKKSTNCAYDRIVLRGQEIVSSVV
PKSNSVFDFQKAYKLTEEEALDVSDHFPVEFKLQSSRAFTNSKKSVTLRKKTKSKRSGGGGS
GGGGSDKTHTSPPSPAPELLGGPSVFLFPPKPKDTLYITREPEVTCVVVDVSHEDPEVKFNW
YVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK
SEQ ID NO: 24
MSRELAPLLLLLLSIHSALAMRICSFNVRSFGESKQEDKNAMDVIVKVIKRCDIILVMEIKD
SNNRICPILMEKLNRNSRRGITYNYVISSRLGRKTYKEQYAFLYKEKLVSVKRSYHYHDYQD
GDADVFSREPFVVWFQSPHTAVKDFVIIPLHTTPETSVKEIDELVEVYTDVKHRWKAENFIF
MGDFNAGCSYVPKKAWKNIRLRTDPRFVWLIGDQEDTTVKKSTNCAYDRIVLRGQEIVSSVV
PKSNSVFDFQKAYKLTEEEALDVSDHFPVEFKLQSSRAFTNSKKSVTLRKKTKSKRSELKTP
LGDTTHTXPRZPAPEFLGGPDKTHTXPPZPAPELLGGPSVFLFPPKPKDTLYITREPEVTCV
VVDVSHEDPEVKFNWYVDGVEVHNAKTKPREEQYNSTYRVVSVLTVLHQDWLNGKEYKCKVS
NKALPAPIEKTISKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHYTQKSLSLSPGK*

wherein each occurrence of X and Z is independently Cys, Gly, or Ser.

In certain non-limiting embodiments, wherein at least one of X and Z is not Cys (C) formation of disulfide bridge is prevented.

SEQ ID NO: 25
MSRELAPLLLLLLSIHSALAMRICSFNVRSF
GESKQEDKNAMDVIVKVIKRCDIILVMEIKD
SNNRICPILMEKLNRNSRRGITYNYVISSRL
GRKTYKEQYAFLYKEKLVSVKRSYHYHDYQD
GDADVFSREPFVVWFQSPHTAVKDFVIIPLH
TTPETSVKEIDELVEVYTDVKHRWKAENFIF
MGDFNAGCSYVPKKAWKNIRLRTDPRFVWLI
GDQEDTTVKKSTNCAYDRIVLRGQEIVSSVV
PKSNSVFDFQKAYKLTEEEALDVSDHFPVEF
KLQSSRAFTNSGSDKTHTXPPZPAPELLGGP
SVFLFPPKPKDTLYITREPEVTCVVVDVSHE
DPEVKFNWYVDGVEVHNAKTKPREEQYNSTY
RVVSVLTVLHQDWLNGKEYKCKVSNKALPAP
IEKTISKAKGQPREPQVYTLPPSREEMTKNQ
VSLTCLVKGFYPSDIAVEWESNGQPENNYKT
TPPVLDSDGSFFLYSKLTVDKSRWQQGNVFS
CSVMHEALHNHYTQKSLSLSPGK

wherein X and Z are independently C, G, or S.

In certain non-limiting embodiments, wherein at least one of X and Z is not Cys (C) formation of disulfide bridge is prevented.

SEQ ID NO: 26
MSRELAPLLLLLLSIHSALAMRICSFNVRSFG [symbol] SKQEDKNA [symbol] DVIVKVIKRCDIILVMEIKD
SNNRICPILMEKLNRNSRRGITYNYVISSRLGRKTYKEQYAFLYKEKLVSVKRSYHYHDYQD
GDADVFSREPFVVWFQSPHTAVKDFVIIPLHTTPETSVKEIDELVEVYTDVKHRWKAENFIF
MGDFNAGCSYVPKKAWKNIRLRTDPRFVWLIGDQEDTTVKKSTNCAYDRIVLRGQEIVSSVV
PKSNSVFDFQKAYKLTEEEALDVSDHFPVEFKLQSSRAFTNSKKSVTLRKKTKSKRSGSCDK
THTCPPCPAPELLGGPSVFLFPPKPKDTLYITREPEVTCVVVDVSHEDPEVKFNWYVDGVEV
HNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGK*
SEQ ID NO: 27
MSRELAPLLLLLLSIHSALAMRICSFNVRSFG [symbol] SKQEDKNA [symbol] DVIVKVIKRCDIILVMEIKD
SNNRICPILMEKLNRNSRRGITYNYVISSRLGRKTYKEQYAFLYKEKLVSVKRSYHYHDYQD
GD [symbol] D [symbol] FSREPFVVWFQSPHTAVKDFVIIPLHTTPETSVKEIDELVEVYTDVKHRWKAENFIF
MGDFNAGCSYVPKKAWKNIRLRTDPRFVWLIGDQEDTTVKKSTNCAYDRIVLRGQEIVSSVV
PKSNSVFDFQKAYKLTEEEALDVSDHFPVEFKLQSSRAFTNSKKSVTLRKKTKSKRSGSCDK
THTCPPCPAPELLGGPSVFLFPPKPKDTLYITREPEVTCVVVDVSHEDPEVKFNWYVDGVEV
HNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGK
SEQ ID NO: 28
MSRELAPLLLLLLSIHSALAMRICSFNVRSFG [symbol] SKQEDKNA [symbol] DVIVKVIKRCDIILVMEIKD
SNNRICPILMEKLNRNSRRGITYNY [symbol] ISSRLGRKTYKEQYAFLYKEKLVSVKRSYHYHDYQD
GDADVFSREPFVVWFQSPHTAVKDFVIIPLHTTPETSVKEIDELVEVYTDVKHRWKAENFIF
MGDFNAGCSYVPKKAWKNIRLRTDPRFVWLIGDQEDTTVKKSTNCAYDRIVLRGQEIVSSVV
PKSNSVFDFQKAYKLTEEEALDVSDHFPVEFKLQSSRAFTNSKKSVTLRKKTKSKRSGSCDK
THTCPPCPAPELLGGPSVFLFPPKPKDTLYITREPEVTCVVVDVSHEDPEVKFNWYVDGVEV
HNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGK
(Sequence1171-mouse DNAse1 construct)
SEQ ID NO: 32
MRYTGLMGTLLTLVNLLQLAGTLRIAAFNIRTFGETKMSNATLSVYFVKILSRYDIAVIQEV
RDSHLVAVGKLLDELNRDKPDTYRYVVSEPLGRKSYKEQYLFVYRPDQVSILDSYQYDDGCE
CGNDTFSREPAIVKFFSPYTEVQEFAIVPLHAAPTEAVSEIDALYDVYLDVWQKWGLEDIMF
MGDFNAGCSYVTSSQWSSIRLRTSPIFQWLIPDSADTTVTSTHCAYDRIVVAGALLQAAVVP
NSAVPFDFQAEYGLSNQLAEAISDHYPVEVTLRKISSTMVRSGCKPCICTVPEVSSVFIFPP
KPKDVLYITLEPKVTCVVVDISKDDPEVQFSWFVDDVEVHTAQTQPREEQFNSTFRSVSELP
IMHQDWLNGKEFKCRVNSAAFPAPIEKTISKTKGRPKAPQVYTIPPPKEQMAKDKVSLTCMI
TDFFPEDITVEWQWNGQPAENYKNTQPIMDTDGSYFVYSKLNVQKSNWEAGNTFTCSVLHEG
LHNHHTEKSLSHSPGK
(Sequence 1671-mouse DNAse1 construct)
SEQ ID NO: 33
MRYTGLMGTLLTLVNLLQLAGTLRIAAFNIRTFGETKMSNATLSVYFVKILSRYDIAVIQEV
RDSHLVAVGKLLDELNRDKPDTYRYVVSEPLGRKSYKEQYLFVYRPDQVSILDSYQYDDGCE
PCGNDTFSREPFIVKFFSPYTEVQEFAIVPLHAAPTEAVSEIDALYDVYLDVWQKWGLEDIM
FMGDFNAGCSYVTSSQWSSIRLRTSPIFQWLIPDSADTTVTSTHCAYDRIVVAGALLQAAVV
PNSAVPFDFQAEYGLSNQLAEAISDHYPVEVTLRKISSTMVGSGCKPCICTVPEVSSVFIFP
PKPKDVLYITLEPKVTCVVVDISKDDPEVQFSWFVDDVEVHTAQTQPREEQFNSTFRSVSEL
PIMHQDWLNGKEFKCRVNSAAFPAPIEKTISKTKGRPKAPQVYTIPPPKEQMAKDKVSLTCM
ITDFFPEDITVEWQWNGQPAENYKNTQPIMDTDGSYFVYSKLNVQKSNWEAGNTFTCSVLHE
GLHNHHTEKSLSHSPGK
(Sequence 1687-mouse DNAse1 construct)
SEQ ID NO: 34
MRYTGLMGTLLTLVNLLQLAGTLRIAAFNIRTFGRTKMSNATLSVYFVKILSRYDIAVIQEV
RDSHLVAVGKLLDELNRDKPDTYRYNVSEPLGRKSYKEQYLFVYRPDQVSILDSYQYDDGCE
PCGNDTFSREPAIVKFFSPYTEVQEFAIVPLHAAPTEAVSEIDALYDVYLDVWQKWGLEDIM
FMGDFNAGCSYVTSSQWSSIRLRTSPIFQWLIPDSADTTVTSTHCAYDRIVVAGALLQAAVV
PNSAVPFDFQAEYGLSNQLAEAISDHYPVEVTLRKISSTMVGSGCKPCICTVPEVSSVFIFP
PKPKDVLYITLEPKVTCVVVDISKDDPEVQFSWFVDDVEVHTAQTQPREEQFNSTFRSVSEL
PIMHQDWLNGKEFKCRVNSAAFPAPIEKTISKTKGRPKAPQVYTIPPPKEQMAKDKVSLTCM
ITDFFPEDITVEWQWNGQPAENYKNTQPIMDTDGSYFVYSKLNVQKSNWEAGNTFTCSVLHE
GLHNHHTEKSLSHSPGK
(Sequence 1689-mouse DNAse1 construct)
SEQ ID NO: 35
MRYTGLMGTLLTLVNLLQLAGTLRIAAFNIRTFGRTKMSNATLSVYFVKILSRYDIAVIQEV
RDSHLVAVGKLLDELNRDKPDTYRYNVSEPLGRKSYKEQYLFVYRPDQVSILDSYQYDDGCE
PCGNDTFSREPAIVKFFSPYTEVQEFAIVPLHAAPTEAVSEIDALYDVYLDVWQKWGLEDIM
FMGDFNAGCSYVTSSQWSSIRLRTSPIFQWLIPDSADTTVTSTHCAYDRIVVAGALLQAAVV
PNSAVPFDFQAEYGLSNQTAEAISDHYPVEVTLRKISSTMVGSGCKPCICTVPEVSSVFIFP
PKPKDVLYITLEPKVTCVVVDISKDDPEVQFSWFVDDVEVHTAQTQPREEQFNSTFRSVSEL
PIMHQDWLNGKEFKCRVNSAAFPAPIEKTISKTKGRPKAPQVYTIPPPKEQMAKDKVSLTCM
ITDFFPEDITVEWQWNGQPAENYKNTQPIMDTDGSYFVYSKLNVQKSNWEAGNTFTCSVLHE
GLHNHHTEKSLSHSPGK
(Sequence 1584-mouse DNAse1L3 construct)
SEQ ID NO: 36
MSLHPASPRLASLLLFILALHDTLALRLCSFNVRSFGASKKENHEAMDIIVKIIKRCDLILL
MEIKDSSNNICPMLMEKLNGNSRRSTTYNYVISSRLGRNTYKEQYAFVYKEKLVSVKTKYHY
HDYQDGDTDVFSREPFVVWFHSPFTAVKDFVIVPLHTTPETSVKEIDELVDVYTDVRSQWKT
ENFIFMGDFNAGCSYVPKKAWQNIRLRTDPKFVWLIGDQEDTTVKKSTSCAYDRIVLCGQEI
VNSVVPRSSGVFDFQKAYDLSEEEALDVSDHFPVEFKLQSSRAFTNNRKSVSLKKRKKGNRS
SSTMVGSGCKPCICTVPEVSSVFIFPPKPKDVLYITLEPKVTCVVVDISKDDPEVQFSWFVD
DVEVHTAQTQPREEQFNSTFRSVSELPIMHQDWLNGKEFKCRVNSAAFPAPIEKTISKTKGR
PKAPQVYTIPPPKEQMAKDKVSLTCMITDFFPEDITVEWQWNGQPAENYKNTQPIMDTDGSY
FVYSKLNVQKSNWEAGNTFTCSVLHEGLHNHHTEKSLSHSPGK
(Sequence 1596-mouse DNAse1L3 construct)
SEQ ID NO: 37
MSLHPASPRLASLLLFILALHDTLALRLCSFNVRSFGASKKENHEAMDIIVKIIKRCDLILL
MEIKDSSNNICPMLMEKLNGNSRRSTTYNYVISSRLGRNTYKEQYAFVYKEKLVSVKTKYHY
HDYQDGDTDVFSREPFVVWFHSPFTAVKDFVIVPLHTTPETSVKEIDELVDVYTDVRSQWKT
ENFIFMGDFNAGCSYVPKKAWQNIRLRTDPKFVWLIGDQEDTTVKKSTSCAYDRIVLCGQEI
VNSVVPRSSGVFDFQKAYDLSEEEALDVSDHFPVEFKLQSSRAFTNNRSGCKPCICTVPEVS
SVFIFPPKPKDVLYITLEPKVTCVVVDISKDDPEVQFSWFVDDVEVHTAQTQPREEQFNSTF
RSVSELPIMHQDWLNGKEFKCRVNSAAFPAPIEKTISKTKGRPKAPQVYTIPPPKEQMAKDK
VSLTCMITDFFPEDITVEWQWNGQPAENYKNTQPIMDTDGSYFVYSKLNVQKSNWEAGNTFT
CSVLHEGLHNHHTEKSLSHSPGK
(Sequence 1615-mouse DNAse1L3 construct)
SEQ ID NO: 38
MSLHPASPRLASLLLFILALHDTLALRLCSFNVRSFGRSKKENHEAMDIIVKIIKRCDLILL
MEIKDSSNNICPMLMEKLNGNSRRSTTYNYVISSRLGRKTYKEQYAFVYKEKLVSVKTKYHY
HDYQDGDTDVFSREPFVVWFHSPFTAVKDFVIVPLHTTPETSVKEIDELVDVYTDVRSQWKT
ENFIFMGDFNAGCSYVPKKAWQNIRLRTDPKFVWLIGDQEDTTVKKSTSCAYDRIVLCGQEI
VNSVVPRSSGVFDFQKAYDLSEEEALDVSDHFPVEFKLQSSRAFTNNRKSVSLKKRKKGNRS
SSTMVGSGCKPCICTVPEVSSVFIFPPKPKDVLYITLEPKVTCVVVDISKDDPEVQFSWFVD
DVEVHTAQTQPREEQFNSTFRSVSELPIMHQDWLNGKEFKCRVNSAAFPAPIEKTISKTKGR
PKAPQVYTIPPPKEQMAKDKVSLTCMITDFFPEDITVEWQWNGQPAENYKNTQPIMDTDGSY
FVYSKLNVQKSNWEAGNTFTCSVLHEGLHNHHTEKSLSHSPGK
(Sequence 1669-mouse DNAse1L3 construct)
SEQ ID NO: 39
MSLHPASPRLASLLLFILALHDTLALRLCSFNVRSFGRSKKENHEAMDIIVKIIKRCDLILL
MEIKDSSNNICPMLMEKLNGNSRRSTTYNYVISSRLGRKTYKEQYAFVYKEKLVSVKTKYHY
HDYQDGDTDVFSREPFVVWFHSPFTAVKDFVIVPLHTTPETSVKEIDELVDVYTDVRSQWKT
ENFIFMGDFNAGCSYVPKKAWQNIRLRTDPKFVWLIGDQEDTTVKKSTSCAYDRIVLCGQEI
VNSVVPRSNGTFDFQKAYDLSEEEALDVSDHFPVEFKLQSSRAFTNNRKSVSLKKRKKGNRS
SSTMVGSGCKPCICTVPEVSSVFIFPPKPKDVLYITLEPKVTCVVVDISKDDPEVQFSWFVD
DVEVHTAQTQPREEQFNSTFRSVSELPIMHQDWLNGKEFKCRVNSAAFPAPIEKTISKTKGR
PKAPQVYTIPPPKEQMAKDKVSLTCMITDFFPEDITVEWQWNGQPAENYKNTQPIMDTDGSY
FVYSKLNVQKSNWEAGNTFTCSVLHEGLHNHHTEKSLSHSPGK

In certain embodiments, the present disclosure contemplates a construct that is expressed from a mammalian cell line, such as but not limited to a CHO cell line, which is stably transfected with human ST6 beta-galactosamide alpha-2,6-sialyltransferase (ST6GAL1). In certain embodiments, such expression enhances sialyation of the construct. The present disclosure further provides a construct that is grown in a cell culture supplemented with sialic acid and/or N-acetylmannosamine (1,3,4-O-Bu3ManNAc). In certain embodiments, such growth enhances sialic acid capping of the construct.

In certain embodiments, enhancing protein sialyation by expressing the biologic in CHO cells stably transfected with human alpha-2,6-sialyltransferase substantially improved construct bioavailability (Cₘₐₓ) when dosed subcutaneously. In other embodiments, increasing the pH-dependent FcRn-mediated cellular recycling by manipulating the Fc domain led to improvements of in vivo biologic half-life. In yet other embodiments, combining CHO cells stably transfected with human α-2,6-sialyltransferase and growing the cells in N-acetylmannosamine led to dramatic increases half-life and/or biologic exposure (AUC). In yet other embodiments, combining two or more methods described herein into a single construct led to dramatic increases in half-life and/or biologic exposure (AUC).

In certain embodiments, the constructs of the disclosure are more highly glycosylated than other DNAse1 and/or DNAse1L3 constructs in the art. In other embodiments, the constructs of the disclosure have higher affinity for the neonatal orphan receptor (FcRn) than other DNAse1 and/or DNAse1L3 constructs in the art. In yet other embodiments, the constructs of the disclosure have higher in vivo half-lives than other DNAse1 and/or DNAse1L3 constructs in the art. In yet other embodiments, the in vivo half-life of a construct of the disclosure is at least about 1.5, 2, 2.5, 3, 4, 5, 6, 7, 8, 9, 10, 12, 14, 16, 18, or 20 times higher than the DNAse1 and/or DNAse1L3 constructs described in the art. In yet other embodiments, the constructs of the disclosure are administered to the subject at a lower dose and/or at a lower frequency than other DNAse1 and/or DNAse1L3 constructs in the art. In yet other embodiments, the constructs of the disclosure are administered to the subject once a month, twice a month, three times a month, and/or four times a month. In yet other embodiments, the lower frequency administration of the constructs of the disclosure results in better patient compliance and/or increased efficacy as compared with other DNAse1 and/or DNAse1L3 constructs in the art.

In certain embodiments, the construct is soluble. In other embodiments, the construct is a recombinant polypeptide.

In certain embodiments, the construct comprises a signal peptide resulting in the secretion of a precursor of the DNAse1 and/or DNAse1L3 polypeptide, which undergoes proteolytic processing to yield a processed construct comprising the DNAse1 and/or DNAse1L3 polypeptide.

In certain embodiments, the DNAse1 and/or DNAse1L3 polypeptide is C-terminally fused to the Fc domain of human immunoglobulin 1 (IgG1), human immunoglobulin 2 (IgG2), human immunoglobulin 3 (IgG3), and/or human immunoglobulin 4 (IgG4). In other embodiments, the DNAse1 and/or DNAse1L3 polypeptide is N-terminally fused to the Fc domain of human immunoglobulin 1 (IgG1), human immunoglobulin 2 (IgG2), human immunoglobulin 3 (IgG3), and/or human immunoglobulin 4 (IgG4). In yet other embodiments, the presence of IgFc domain improves half-life, solubility, reduces immunogenicity, and increases the activity of the DNAse1 and/or DNAse1L3 polypeptide.

In certain embodiments, the DNAse1 and/or DNAse1L3 polypeptide is C-terminally fused to human serum albumin. Human serum albumin may be conjugated to DNAse1 and/or DNAse1L3 protein through a chemical linker, including but not limited to naturally occurring or engineered disulfide bonds, and/or by genetic fusion to DNAse1 and/or DNAse1L3, and/or a fragment and/or variant thereof.

In certain embodiments, the construct is further pegylated (i.e., fused with a poly(ethylene glycol) chain).

In certain embodiments, the construct is formulated as a liquid formulation. In other embodiments, the disclosure provides a dry product form of a pharmaceutical composition comprising a therapeutic amount of a construct of the disclosure, whereby the dry product is reconstitutable to a solution of the construct in liquid form.

The disclosure provides a kit comprising at least one construct of the disclosure, and/or a salt or solvate thereof, and instructions for using the construct within the methods of the disclosure.

It will be understood that a DNAse1 and/or DNAse1L3 polypeptide according to the disclosure includes not only the native human proteins, but also any fragment, derivative, fusion, conjugate or mutant thereof. As used herein in this disclosure, the phrase “a DNAse1 and/or DNAse1L3 polypeptide, mutant, and/or mutant fragment thereof” also includes any compound or polypeptide (such as, but not limited to, a fusion protein) comprising a DNAse1 and/or DNAse1L3 polypeptide, mutant, and/or mutant fragment thereof. Fusion proteins according to the disclosure are considered biological equivalents of DNAse1 and/or DNAse1L3, but can in certain embodiments provide longer half-life or greater potency due to increased in vivo biologic exposure, as judged by the “area under the curve” (AUC) or increased half-life in pharmacokinetic experiments.

### Vectors and Cells

The disclosure further provides an autonomously replicating or an integrative mammalian cell vector comprising a recombinant nucleic acid encoding a polypeptide of the disclosure. In certain embodiments, the vector comprises a plasmid or a virus. In other embodiments, the vector comprises a mammalian cell expression vector. In yet other embodiments, the vector further comprises at least one nucleic acid sequence that directs and/or controls expression of the polypeptide. In yet other embodiments, the recombinant nucleic acid encodes a construct comprising a DNAse1 and/or DNAse1L3 polypeptide and a signal peptide, wherein the polypeptide is proteolytically processed upon secretion from a cell to yield the DNAse1 and/or DNAse1L3 construct of the disclosure.

In yet another aspect, the disclosure provides an isolated host cell comprising a vector of the disclosure. In certain embodiments, the cell is a non-human cell. In other embodiments, the cell is mammalian. In yet other embodiments, the vector of the disclosure comprises a recombinant nucleic acid encoding a construct comprising a DNAse1 and/or DNAse1L3 polypeptide and a signal peptide. In yet other embodiments, the polypeptide is proteolytically processed upon secretion from a cell to yield the DNAse1 and/or DNAse1L3 construct of the disclosure.

Production and Purification of DNAse1 and/or DNAse1L3 Fusion Proteins

In certain embodiments, a soluble DNAse1 and/or DNAse1L3 construct, including IgG Fc domain or enzymatically/biologically active fragments thereof, are efficacious in treating, reducing, and/or preventing progression of diseases or disorders contemplated herein.

To produce soluble, recombinant DNAse1 and/or DNAse1L3 constructs for in vitro use, DNAse1 and/or DNAse1L3 polypeptides can be fused to the Fc domain of IgG (referred to as “DNAse1-Fc” or “DNAse1L3-Fc”) and the fusion construct can be expressed in stable CHO cell lines. The construct can also be expressed from HEK293 cells, Baculovirus insect cell system or CHO cells or Yeast Pichia expression system using suitable vectors. The construct can be produced in either adherent or suspension cells. To establish stable cell lines the nucleic acid sequence encoding DNAse1 and/or DNAse1L3 constructs are cloned into an appropriate vector for large scale protein production.

Many expression systems are known can be used for the production of DNAse1 and/or DNAse1L3 constructs, including bacteria (for example E. coli and Bacillus subtilis), yeasts (for example Saccharomyces cerevisiae, Kluyveronmyces lactis and Pichia pastoris), filamentous fungi (for example Aspergillus), plant cells, animal cells and insect cells. The desired proteins can be produced in conventional ways, for example from a coding sequence inserted in the host chromosome or on a free plasmid.

The yeasts can be transformed with a coding sequence for the desired protein in any one of the usual ways, for example electroporation. Methods for transformation of yeast by electroporation are disclosed in Becker & Guarente, 1990, Methods Enzymol. 194: 182. Successfully transformed cells, i.e., cells that contain a DNA construct of the present disclosure, can be identified by well-known techniques. For example, cells resulting from the introduction of an expression construct can be grown to produce the desired polypeptide. Cells can be harvested and lysed and their DNA content examined for the presence of the DNA using a method, such as that described by Southern, 1975, J. Mol. Biol, 98:503 and/or Berent, et al., 1985, Biotech 3:208. Alternatively, the presence of the protein in the supernatant can be detected using antibodies.

Useful yeast plasmid vectors include pRS403-406 and pRS413-416 and are generally available fron1 Strat:1.gene Cloning Systems, La Jolla, Calif., USA Plasmids pRS403, pRS404, pRS405 and pRS406 are Yeast Integrating plasmids (Y1ps) and incorporate the yeast selectable markers I-llS3, TRP1, LEU2 and lJRA3. Plasmids pRS413-416 are Yeast Centromere plasmids (YCps).

A variety of methods have been developed to operably link DNA to vectors via complementary cohesive termini. For instance, complementary homopolymer tract can be added to the DNA segment to be inserted to the vector DNA. The vector and DNA segment are then joined by hydrogen bonding between the complementary homopolymeric tails to form recombinant DNA molecules.

Synthetic linkers containing one or more restriction sites provide an alternative method of joining the DNA segment to vectors. The DNA segment, generated by endonuclease restriction digestion, is treated with bacteriophage T4 DNA polymerase or E. coli DNA polymerase I, which are enzymes that remove protruding, 3′-single-stranded termini with their 3′-5′-exonucleolytic activities, and fill in recessed 3′-ends with their polymerizing activities.

The combination of these activities thus generates blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the products of the reaction are DNA segments carrying polymeric linker sequences at their ends. These DNA segments are then cleaved with the appropriate restriction enzyme and ligated to an expression vector that has been cleaved with an enzyme that produces termini compatible with those of the DNA segment.

Clones of single, stably transfected cells are then established and screened for high expressing clones of the desired fusion protein. Screening of the single cell clones for DNAse1 and/or DNAse1L3 protein expression can be accomplished in a high-throughput manner in 96 well plates. Upon identification of high expressing clones through screening, protein production can be accomplished in shaking flasks or bio-reactors.

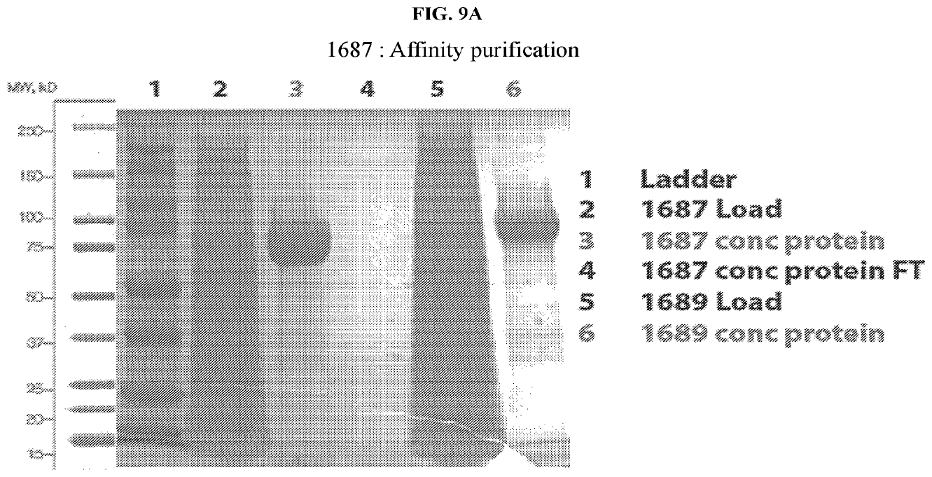

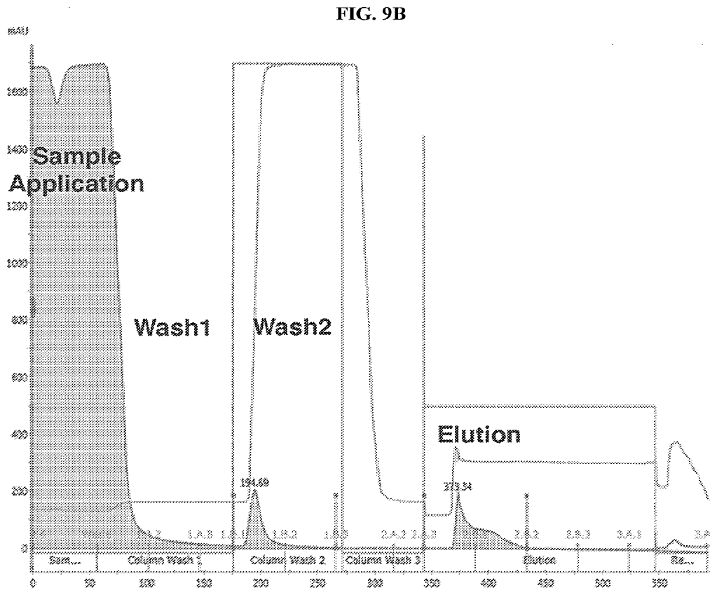

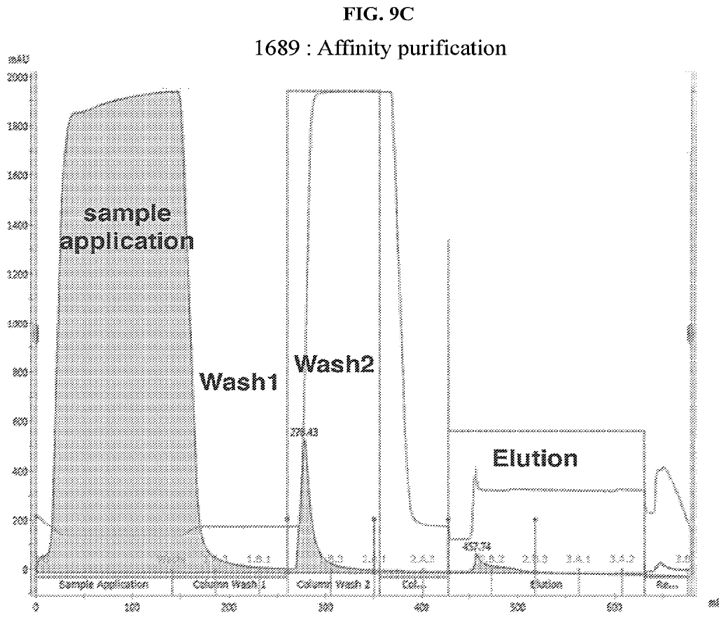

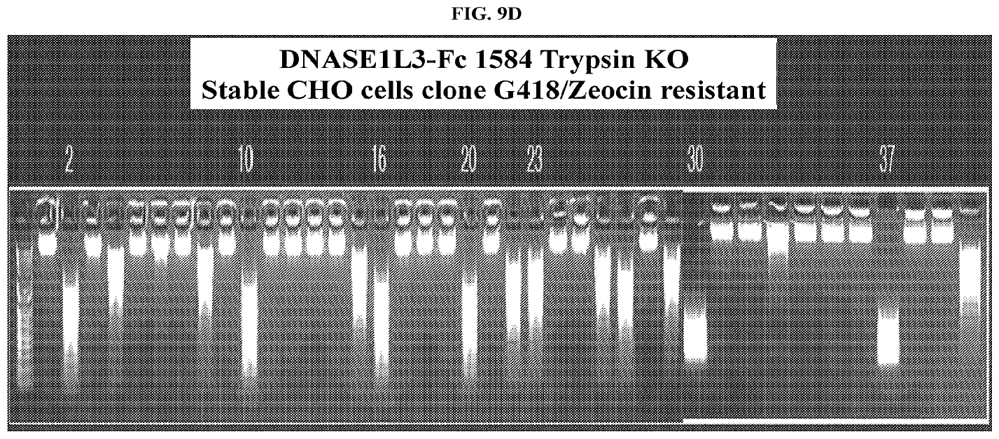

Purification of DNAse1 and/or DNAse1L3 constructs can be accomplished using a combination of standard purification techniques known in the art. Annotated examples of affinity purifications of some of the constructs proposed are provided in FIGS. 9A-9D.

### Gene Therapy

The nucleic acids encoding the polypeptide(s) useful within the disclosure may be used in gene therapy protocols for the treatment of the diseases or disorders contemplated herein. The improved construct encoding the polypeptide(s) can be inserted into the appropriate gene therapy vector and administered to a patient to treat or prevent the diseases or disorder of interest.

Vectors, such as viral vectors, have been used in the prior art to introduce genes into a wide variety of different target cells. Typically the vectors are exposed to the target cells so that transformation can take place in a sufficient proportion of the cells to provide a useful therapeutic or prophylactic effect from the expression of the desired polypeptide (e.g., a receptor). The transfected nucleic acid may be permanently incorporated into the genome of each of the targeted cells, providing long lasting effect, or alternatively the treatment may have to be repeated periodically. In certain embodiments, the (viral) vector transfects liver cells in vivo with genetic material encoding the polypeptide(s) of the disclosure.

A variety of vectors, both viral vectors and plasmid vectors are known in the art (see for example U.S. Pat. No. 5,252,479 and WO 93/07282). In particular, a number of viruses have been used as gene transfer vectors, including papovaviruses, such as SV40, vaccinia virus, herpes viruses including HSV and EBV, and retroviruses. Many gene therapy protocols in the prior art have employed disabled murine retroviruses. Several recently issued patents are directed to methods and compositions for performing gene therapy (see for example U.S. Pat. Nos. 6,168,916; 6,135,976; 5,965,541 and 6,129,705). Each of the foregoing patents is incorporated by reference in its entirety herein.

### AAV-Mediated Gene Therapy:

AAV, a parvovirus belonging to the genus Dependovirus, has several features that make it particularly well suited for gene therapy applications. For example, AAV can infect a wide range of host cells, including non-dividing cells. Furthermore, AAV can infect cells from a variety of species. Importantly, AAV has not been associated with any human or animal disease, and does not appear to alter the physiological properties of the host cell upon integration. Finally, AAV is stable at a wide range of physical and chemical conditions, which lends itself to production, storage, and transportation requirements.

The AAV genome, which is a linear, single-stranded DNA molecule containing approximately 4,700 nucleotides (the AAV-2 genome consists of 4,681 nucleotides, the AAV-4 genome 4,767), generally comprises an internal non-repeating segment flanked on each end by inverted terminal repeats (ITRs). The ITRs are approximately 145 nucleotides in length (AAV-1 has ITRs of 143 nucleotides) and have multiple functions, including serving as origins of replication, and as packaging signals for the viral genome.

The internal non-repeated portion of the genome includes two large open reading frames (ORFs), known as the AAV replication (rep) and capsid (cap) regions. These ORFs encode replication and capsid gene products, which allow for the replication, assembly, and packaging of a complete AAV virion. More specifically, a family of at least four viral proteins are expressed from the AAV rep region: Rep 78, Rep 68, Rep 52, and Rep 40, all of which are named for their apparent molecular weights. The AAV cap region encodes at least three proteins: VP1, VP2, and VP3.

AAV is a helper-dependent virus, that is, it requires co-infection with a helper virus (e.g., adenovirus, herpesvirus, or vaccinia virus) in order to form functionally complete AAV virions. In the absence of co-infection with a helper virus, AAV establishes a latent state in which the viral genome inserts into a host cell chromosome or exists in an episomal form, but infectious virions are not produced. Subsequent infection by a helper virus “rescues” the integrated genome, allowing it to be replicated and packaged into viral capsids, thereby reconstituting the infectious virion. While AAV can infect cells from different species, the helper virus must be of the same species as the host cell. Thus, for example, human AAV replicates in canine cells that have been co-infected with a canine adenovirus.

To produce infectious recombinant AAV (rAAV) containing a heterologous nucleic acid sequence, a suitable host cell line can be transfected with an AAV vector containing the heterologous nucleic acid sequence, but lacking the AAV helper function genes, rep and cap. The AAV-helper function genes can then be provided on a separate vector. Also, only the helper virus genes necessary for AAV production (i.e., the accessory function genes) can be provided on a vector, rather than providing a replication-competent helper virus (such as adenovirus, herpesvirus, or vaccinia).

Collectively, the AAV helper function genes (i.e., rep and cap) and accessory function genes can be provided on one or more vectors. Helper and accessory function gene products can then be expressed in the host cell where they will act in trans on rAAV vectors containing the heterologous nucleic acid sequence. The rAAV vector containing the heterologous nucleic acid sequence will then be replicated and packaged as though it were a wild-type (wt) AAV genome, forming a recombinant virion. When a patient's cells are infected with the resulting rAAV virions, the heterologous nucleic acid sequence enters and is expressed in the patient's cells. Because the patient's cells lack the rep and cap genes, as well as the accessory function genes, the rAAV cannot further replicate and package their genomes. Moreover, without a source of rep and cap genes, wtAAV cannot be formed in the patient's cells.

There are eleven known AAV serotypes, AAV-1 through AAV-11 (Mori, et al., 2004, Virology 330(2):375-83). AAV-2 is the most prevalent serotype in human populations; one study estimated that at least 80% of the general population has been infected with wt AAV-2 (Berns and Linden, 1995, Bioessays 17:237-245). AAV-3 and AAV-5 are also prevalent in human populations, with infection rates of up to 60% (Georg-Fries, et al., 1984, Virology 134:64-71). AAV-1 and AAV-4 are simian isolates, although both serotypes can transduce human cells (Chiorini, et al., 1997, J Virol 71:6823-6833; Chou, et al., 2000, Mol Ther 2:619-623). Of the six known serotypes, AAV-2 is the best characterized. For instance, AAV-2 has been used in a broad array of in vivo transduction experiments, and has been shown to transduce many different tissue types including: mouse (U.S. Pat. Nos. 5,858,351; 6,093,392), dog muscle; mouse liver (Couto, et al., 1999, Proc. Natl. Acad. Sci. USA 96:12725-12730; Couto, et al., 1997, J. Virol. 73:5438-5447; Nakai, et al., 1999, J. Virol. 73:5438-5447; and, Snyder, et al., 1997, Nat. Genet. 16:270-276); mouse heart (Su, et al., 2000, Proc. Natl. Acad. Sci. USA 97:13801-13806); rabbit lung (Flotte, et al., 1993, Proc. Natl. Acad. Sci. USA 90:10613-10617); and rodent photoreceptors (Flannery et al., 1997, Proc. Natl. Acad. Sci. USA 94:6916-6921).

The broad tissue tropism of AAV-2 may be exploited to deliver tissue-specific transgenes. For example, AAV-2 vectors have been used to deliver the following genes: the cystic fibrosis transmembrane conductance regulator gene to rabbit lungs (Flotte, et al., 1993, Proc. Natl. Acad. Sci. USA 90:10613-10617); Factor NIII gene (Burton, et al., 1999, Proc. Natl. Acad. Sci. USA 96:12725-12730) and Factor IX gene (Nakai, et al., 1999, J. Virol. 73:5438-5447; Snyder, et al., 1997, Nat. Genet. 16:270-276; U.S. Pat. No. 6,093,392) to mouse liver, dog, and mouse muscle (U.S. Pat. No. 6,093,392); erythropoietin gene to mouse muscle (U.S. Pat. No. 5,858,351); vascular endothelial growth factor (VEGF) gene to mouse heart (Su, et al., 2000, Proc. Natl. Acad. Sci. USA 97:13801-13806); and aromatic 1-amino acid decarboxylase gene to monkey neurons. Expression of certain rAAV-delivered transgenes has therapeutic effect in laboratory animals; for example, expression of Factor IX was reported to have restored phenotypic normalcy in dog models of hemophilia B (U.S. Pat. No. 6,093,392). Moreover, expression of rAAV-delivered NEGF to mouse myocardium resulted in neovascular formation (Su, et al., 2000, Proc. Natl. Acad. Sci. USA 97:13801-13806), and expression of rAAV-delivered AADC to the brains of parkinsonian monkeys resulted in the restoration of dopaminergic function.

Delivery of a protein of interest to the cells of a mammal is accomplished by first generating an AAV vector comprising DNA encoding the protein of interest and then administering the vector to the mammal. Thus, the disclosure should be construed to include AAV vectors comprising DNA encoding the polypeptide(s) of interest. Once armed with the present disclosure, the generation of AAV vectors comprising DNA encoding this/these polypeptide(s)s will be apparent to the skilled artisan.

In certain embodiments, the rAAV vector of the disclosure comprises several essential DNA elements. In certain embodiments, these DNA elements include at least two copies of an AAV ITR sequence, a promoter/enhancer element, a transcription termination signal, any necessary 5′ or 3′ untranslated regions which flank DNA encoding the protein of interest or a biologically active fragment thereof. The rAAV vector of the disclosure may also include a portion of an intron of the protein on interest. Also, optionally, the rAAV vector of the disclosure comprises DNA encoding a mutated polypeptide of interest.

In certain embodiments, the vector comprises a promoter/regulatory sequence that comprises a promiscuous promoter which is capable of driving expression of a heterologous gene to high levels in many different cell types. Such promoters include, but are not limited to the cytomegalovirus (CMV) immediate early promoter/enhancer sequences, the Rous sarcoma virus promoter/enhancer sequences and the like. In certain embodiments, the promoter/regulatory sequence in the rAAV vector of the disclosure is the CMV immediate early promoter/enhancer. However, the promoter sequence used to drive expression of the heterologous gene may also be an inducible promoter, for example, but not limited to, a steroid inducible promoter, or may be a tissue specific promoter, such as, but not limited to, the skeletal a-actin promoter which is muscle tissue specific and the muscle creatine kinase promoter/enhancer, and the like.

In certain embodiments, the rAAV vector of the disclosure comprises a transcription termination signal. While any transcription termination signal may be included in the vector of the disclosure, in certain embodiments, the transcription termination signal is the SV40 transcription termination signal.

In certain embodiments, the rAAV vector of the disclosure comprises isolated DNA encoding the polypeptide of interest, or a biologically active fragment of the polypeptide of interest. The disclosure should be construed to include any mammalian sequence of the polypeptide of interest, which is either known or unknown. Thus, the disclosure should be construed to include genes from mammals other than humans, which polypeptide functions in a substantially similar manner to the human polypeptide. Preferably, the nucleotide sequence comprising the gene encoding the polypeptide of interest is about 50% homologous, more preferably about 70% homologous, even more preferably about 80% homologous and most preferably about 90% homologous to the gene encoding the polypeptide of interest.

Further, the disclosure should be construed to include naturally occurring variants or recombinantly derived mutants of wild type protein sequences, which variants or mutants render the polypeptide encoded thereby either as therapeutically effective as full-length polypeptide, or even more therapeutically effective than full-length polypeptide in the gene therapy methods of the disclosure.

The disclosure should also be construed to include DNA encoding variants which retain the polypeptide's biological activity. Such variants include proteins or polypeptides which have been or may be modified using recombinant DNA technology, such that the protein or polypeptide possesses additional properties which enhance its suitability for use in the methods described herein, for example, but not limited to, variants conferring enhanced stability on the protein in plasma and enhanced specific activity of the protein. Analogs can differ from naturally occurring proteins or peptides by conservative amino acid sequence differences or by modifications which do not affect sequence, or by both. For example, conservative amino acid changes may be made, which although they alter the primary sequence of the protein or peptide, do not normally alter its function.

The disclosure is not limited to the specific rAAV vector exemplified in the experimental examples; rather, the disclosure should be construed to include any suitable AAV vector, including, but not limited to, vectors based on AAV-1, AAV-3, AAV-4 and AAV-6, and the like.

Also included in the disclosure is a method of treating a mammal having a disease or disorder in an amount effective to provide a therapeutic effect. The method comprises administering to the mammal an rAAV vector encoding the polypeptide of interest. Preferably, the mammal is a human.

Typically, the number of viral vector genomes/mammal which are administered in a single injection ranges from about 1×10⁸ to about 5×10¹⁶. Preferably, the number of viral vector genomes/mammal which are administered in a single injection is from about 1×10¹⁰ to about 1×10¹⁵; more preferably, the number of viral vector genomes/mammal which are administered in a single injection is from about 5×10¹⁰ to about 5×10¹⁵; and, most preferably, the number of viral vector genomes which are administered to the mammal in a single injection is from about 5×10¹¹ to about 5×10¹⁴.

When the method of the disclosure comprises multiple site simultaneous injections, or several multiple site injections comprising injections into different sites over a period of several hours (for example, from about less than one hour to about two or three hours) the total number of viral vector genomes administered may be identical, or a fraction thereof or a multiple thereof, to that recited in the single site injection method.

For administration of the rAAV vector of the disclosure in a single site injection, in certain embodiments a composition comprising the virus is injected directly into an organ of the subject (such as, but not limited to, the liver of the subject).

For administration to the mammal, the rAAV vector may be suspended in a pharmaceutically acceptable carrier, for example, HEPES buffered saline at a pH of about 7.8. Other useful pharmaceutically acceptable carriers include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The rAAV vector of the disclosure may also be provided in the form of a kit, the kit comprising, for example, a freeze-dried preparation of vector in a dried salts formulation, sterile water for suspension of the vector/salts composition and instructions for suspension of the vector and administration of the same to the mammal.

### Methods

The disclosure includes a method of treating, ameliorating, or preventing inefficient NET hydrolysis (“NETolysis”) in a subject afflicted with a bacterial and/or viral infection. The disclosure further includes a method of treating, ameliorating, or preventing systemic inflammation, organ damage and/or sepsis in a subject afflicted with a bacterial and/or viral infection.

In certain embodiments, the method comprises administering a construct of the disclosure to the subject who is suffering from, suspect of suffering from, and/or likely to develop any disease or disorder contemplated herein.

In certain embodiments, the construct of the disclosure is a secreted product of a DNAse1 and/or DNAse1L3 precursor construct (which is itself a construct contemplated within the disclosure) expressed in a mammalian cell. In other embodiments, the DNAse1 and/or DNAse1L3 precursor construct comprises a signal peptide sequence and a DNAse1 and/or DNAse1L3 polypeptide, wherein the DNAse1 and/or DNAse1L3 precursor construct undergoes proteolytic processing to a processed construct comprising the DNAse1 and/or DNAse1L3 polypeptide. In yet other embodiments, in the DNAse1 and/or DNAse1L3 precursor construct the signal peptide sequence is conjugated to the DNAse1 and/or DNAse1L3 polypeptide N-terminus. Upon proteolysis, the signal sequence is cleaved from the DNAse1 and/or DNAse1L3 precursor construct to provide the construct comprising the DNAse1 and/or DNAse1L3 polypeptide.

In certain embodiments, the construct is administered acutely or chronically to the subject. In other embodiments, the construct is administered locally, regionally, parenterally or systemically to the subject

In certain embodiments, the subject is a mammal. In other embodiments, the mammal is human.

In certain embodiments, the construct, and/or its precursor construct, is administered by at least one route selected from the group consisting of subcutaneous, oral, aerosol, inhalational, rectal, vaginal, transdermal, subcutaneous, intranasal, buccal, sublingual, parenteral, intrathecal, intragastrical, ophthalmic, pulmonary and topical. In other embodiments, the construct, and/or its precursor construct, is administered to the subject as a pharmaceutical composition further comprising at least one pharmaceutically acceptable carrier.

In certain embodiments, the construct, and/or its precursor construct, is administered acutely or chronically to the subject. In other embodiments, the construct, and/or its precursor construct, is administered locally, regionally or systemically to the subject. In yet another embodiment, the construct, and/or its precursor construct, is delivered on an encoded vector, wherein the vector encodes the protein and it is transcribed and translated from the vector upon administration of the vector to the subject.

It will be appreciated by one of skill in the art, when armed with the present disclosure including the methods detailed herein, that the disclosure is not limited to treatment of a disease or disorder once it is established. Particularly, the symptoms of the disease or disorder need not have manifested to the point of detriment to the subject; indeed, the disease or disorder need not be detected in a subject before treatment is administered. That is, significant pathology from disease or disorder does not have to occur before the present disclosure may provide benefit.

Thus, the present disclosure, as described more fully herein, includes a method for preventing diseases and disorders in a subject, in that a polypeptide or construct of the disclosure, as discussed elsewhere herein, can be administered to a subject prior to the onset of the disease or disorder, thereby preventing the disease or disorder from developing. Particularly, where the symptoms of the disease or disorder have not manifested to the point of detriment to the subject; indeed, the disease or disorder need not be detected in a subject before treatment is administered. That is, significant pathology from the disease or disorder does not have to occur before the present disclosure may provide benefit. Therefore, the present disclosure includes methods for preventing or delaying onset, and/or reducing progression or growth, of a disease or disorder in a subject, in that a polypeptide of the disclosure can be administered to a subject prior to detection of the disease or disorder. In certain embodiments, the polypeptide of the disclosure is administered to a subject with a strong family history of the disease or disorder, thereby preventing or delaying onset or progression of the disease or disorder.

Armed with the disclosure herein, one skilled in the art would thus appreciate that the prevention of a disease or disorder in a subject encompasses administering to a subject a polypeptide of the disclosure as a preventative measure against the disease or disorder.

### Pharmaceutical Compositions and Formulations

The disclosure provides pharmaceutical compositions comprising a polypeptide of the disclosure within the methods described herein.

Such a pharmaceutical composition is in a form suitable for administration to a subject, and/or the pharmaceutical composition may further comprise one or more pharmaceutically acceptable carriers, one or more additional ingredients, and/or some combination of these. The various components of the pharmaceutical composition may be present in the form of a physiologically acceptable salt, such as in combination with a physiologically acceptable cation or anion, as is well known in the art.

In an embodiment, the pharmaceutical compositions useful for practicing the method of the disclosure may be administered to deliver a dose of between 1 ng/kg/day and 100 mg/kg/day. In other embodiments, the pharmaceutical compositions useful for practicing the disclosure may be administered to deliver a dose of between 1 ng/kg/day and 500 mg/kg/day.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between about 0.1% and about 100% (w/w) active ingredient.

Pharmaceutical compositions that are useful in the methods of the disclosure may be suitably developed for inhalational, oral, rectal, vaginal, parenteral, topical, transdermal, pulmonary, intranasal, buccal, ophthalmic, intrathecal, intravenous or another route of administration. Other contemplated formulations include projected nanoparticles, liposomal preparations, resealed erythrocytes containing the active ingredient, and immunologically-based formulations. The route(s) of administration is readily apparent to the skilled artisan and depends upon any number of factors including the type and severity of the disease being treated, the type and age of the veterinary or human patient being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

### Administration/Dosing

The regimen of administration may affect what constitutes an effective amount. For example, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, such as a mammal, such as a human, may be carried out using known procedures, at dosages and for periods of time effective to treat a disease or disorder in the patient. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. Dosage is determined based on the biological activity of the therapeutic compound which in turn depends on the half-life and the area under the plasma time of the therapeutic compound curve. The polypeptide according to the disclosure can be administered at an appropriate time interval of every 2 days, or every 4 days, or every week or every month. Therapeutic dosage of the polypeptides of the disclosure may also be determined based on half-life or the rate at which the therapeutic polypeptide is cleared out of the body. The polypeptide according to the disclosure is administered at appropriate time intervals of either every 2 days, or every 4 days, every week or every month so as to achieve a constant level of enzymatic activity of DNAse1 and/or DNAse1L3.

For example, several divided doses may be administered daily or the dose may be proportionally reduced as indicated by the exigencies of the therapeutic situation. A non-limiting example of an effective dose range for a therapeutic compound of the disclosure is from about 0.01 and 50 mg/kg of body weight/per day. In some embodiments, the effective dose range for a therapeutic compound of the disclosure is from about 50 ng to 500 ng/kg, preferably 100 ng to 300 ng/kg of bodyweight. One of ordinary skill in the art would be able to study the relevant factors and make the determination regarding the effective amount of the therapeutic compound without undue experimentation.

The compound can be administered to a patient as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon any number of factors, such as, but not limited to, the type and severity of the disease being treated, and the type and age of the patient.

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

A medical doctor, e.g., physician, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In certain embodiments, the compositions of the disclosure are administered to the patient in dosages that range from one to five times per day or more. In other embodiments, the compositions of the disclosure are administered to the patient in range of dosages that include, but are not limited to, once every day, every two, days, every three days to once a week, and once every two weeks. The frequency of administration of the various combination compositions of the disclosure varies from subject to subject depending on many factors including, but not limited to, age, disease or disorder to be treated, gender, overall health, and other factors. Thus, the disclosure should not be construed to be limited to any particular dosage regime and the precise dosage and composition to be administered to any patient will be determined by the attending physical taking all other factors about the patient into account.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of a disease or disorder in a patient.

### Routes of Administration

Routes of administration of any one of the compositions of the disclosure include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. The formulations and compositions that would be useful in the present disclosure are not limited to the particular formulations and compositions that are described herein.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

### Additional Administration Forms

Additional dosage forms of this disclosure include dosage forms as described in U.S. Pat. Nos. 6,340,475, 6,488,962, 6,451,808, 5,972,389, 5,582,837, and 5,007,790. Additional dosage forms of this disclosure also include dosage forms as described in U.S. Patent Applications Nos. 20030147952, 20030104062, 20030104053, 20030044466, 20030039688, and 20020051820. Additional dosage forms of this disclosure also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

Controlled- or sustained-release formulations of a pharmaceutical composition of the disclosure may be made using conventional technology. In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Single unit dosage forms suitable for oral administration, such as tablets, capsules, gelcaps, and caplets, which are adapted for controlled-release are encompassed by the present disclosure.

In certain embodiments, the formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release that is longer that the same amount of agent administered in bolus form. For sustained release, the compounds may be formulated with a suitable polymer or hydrophobic material that provides sustained release properties to the compounds. As such, the compounds for use the method of the disclosure may be administered in the form of microparticles, for example, by injection or in the form of wafers or discs by implantation. In certain embodiments of the disclosure, the compounds of the disclosure are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours. The term pulsatile release is used herein in its conventional sense to refer to a drug formulation that provides release of the drug in such a way as to produce pulsed plasma profiles of the drug after drug administration. The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction and preparation conditions, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present disclosure as set forth herein.

### EXAMPLES

The disclosure is now described with reference to the following Examples. These Examples are provided for the purpose of illustration only, and the disclosure is not limited to these Examples, but rather encompasses all variations that are evident as a result of the teachings provided herein.

### Methods and Materials

Unless specifically mentioned, expression of constructs in CHO cells or modified CHO cells with and without supplementation, enzymatic assays, AUC assay, half-life assay can be carried out using protocols described elsewhere herein or as known in the prior art.

### Area Under the Curve Assay

The area under the plasma concentration versus time curve, also called the area under the curve (AUC) can be used as a means of evaluating the volume of distribution (V), total elimination clearance (CL), and bioavailability (F) for extravascular drug delivery. Area under plasma time curve for each expressed and purified DNAse1-Fc and/or DNAse1L3-Fc construct can be carried out using the standard equation to determine half-life and bioavailability after a single subcutaneous injection of biologic, as described in Equation 1.

### Half-Life Determination

The drug half-life (t_{1/2}) is the time it takes for the plasma concentration or the amount of drug or biologic in the body to be reduced by 50%. Half-life values for each expressed and purified construct can be carried out following protocols described in the prior art and/or herein, such as Equation 1, which allows for determining half-life and bioavailability after a single subcutaneous injection of biologic.

Drug half-life can be calculated using Equation 1, which correlates the relationship between systemic fractional concentration and time of a drug administered to a subcutaneous depot in a single injection. Plotting the data as fraction of drug absorbed (F) over time (t) allows for the determination of the elimination (kₑ) and absorption (kₐ) constants by fitting the data to the equation for the total systemic absorption of a drug administered at a subcutaneous depot at time t=0.

$$F = \frac{{k}_{a}}{( {k}_{a} - {k}_{e} )} [ {e}^{- {k}_{e} t} - {e}^{- {k}_{a} t} ] \qquad ( Equation 1 )$$

### EXAMPLES

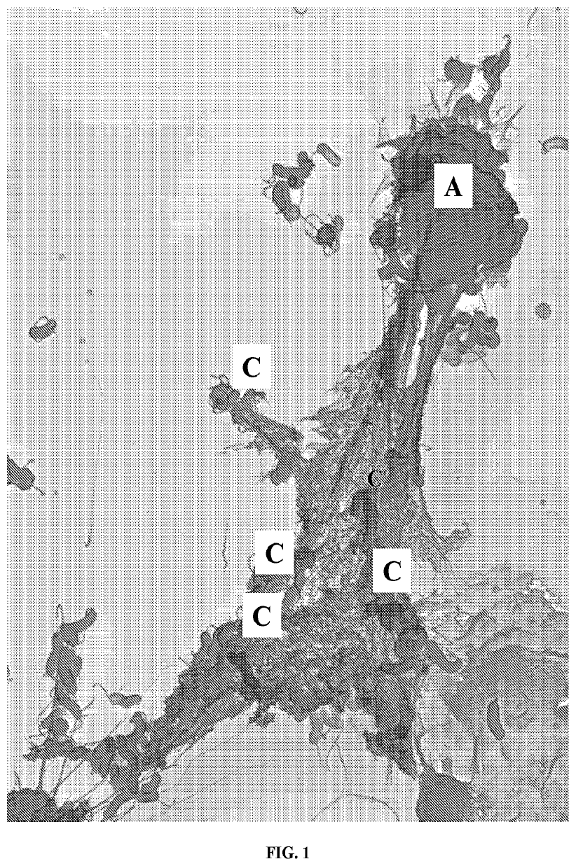

FIG. 1 illustrates neutrophil extracellular trap (NET) formation. Scanning electron microscopy of neutrophil (marked as A) casting a net (marked as B) entrapping Helicobacter pylori bacteria (some of which are marked as C). Image taken from Kumamoto T, et al., 2006, Eur. Heart J. 27(17):2081-7.

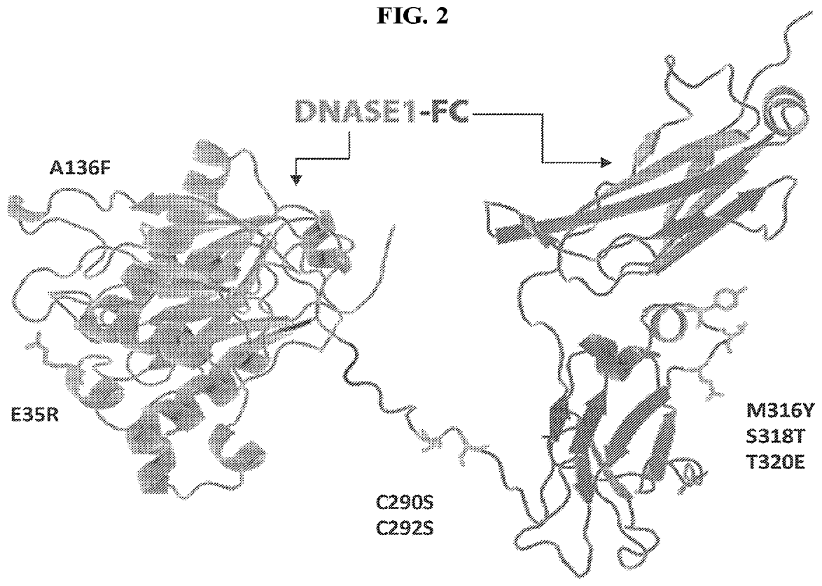

FIG. 2 illustrates a non-limiting DNAse1-Fc construct of the disclosure, with certain contemplated point mutations highlighted.

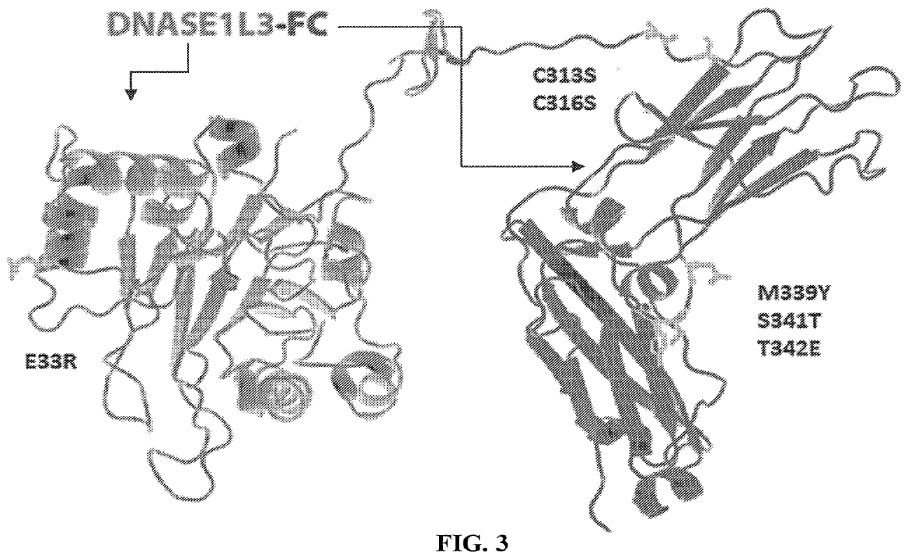

FIG. 3 illustrates a non-limiting DNAse1L3-Fc construct of the disclosure, with certain contemplated point mutations highlighted.

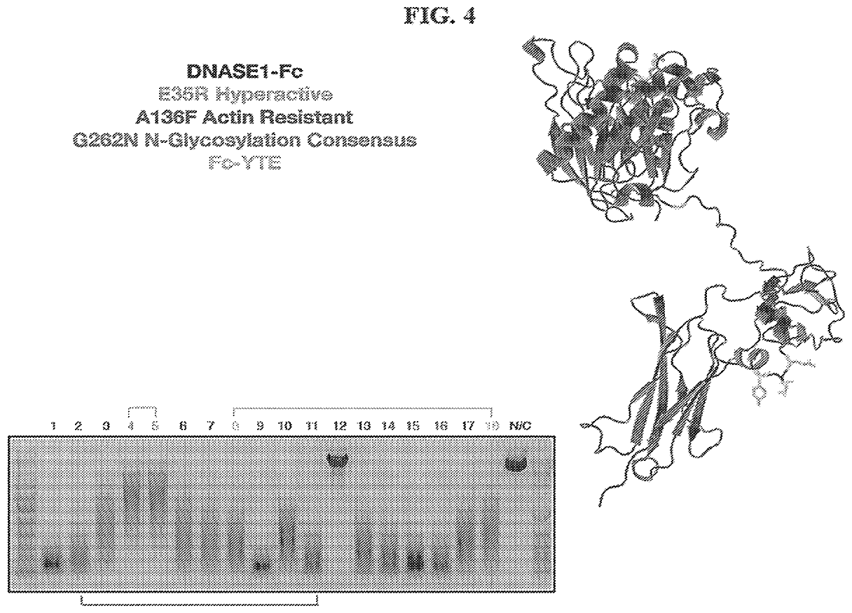

FIG. 4 illustrates a non-limiting DNAse1-Fc construct of the disclosure, with certain contemplated point mutations highlighted.

FIG. 5 illustrates non-limiting constructs of the disclosure, with certain contemplated point mutations highlighted. In certain embodiments, certain mutations render the rDNAse hyperactive and/or render the rDNAse actin-resistant (i.e., has decreased affinity for actin) and/or increase the construct's half-life. The non-limiting aligment of amino acid sequences of mouse DNAse1 (SEQ ID NO:42) and mouse DNAse1L3 (SEQ ID NO:43) is illustrated.

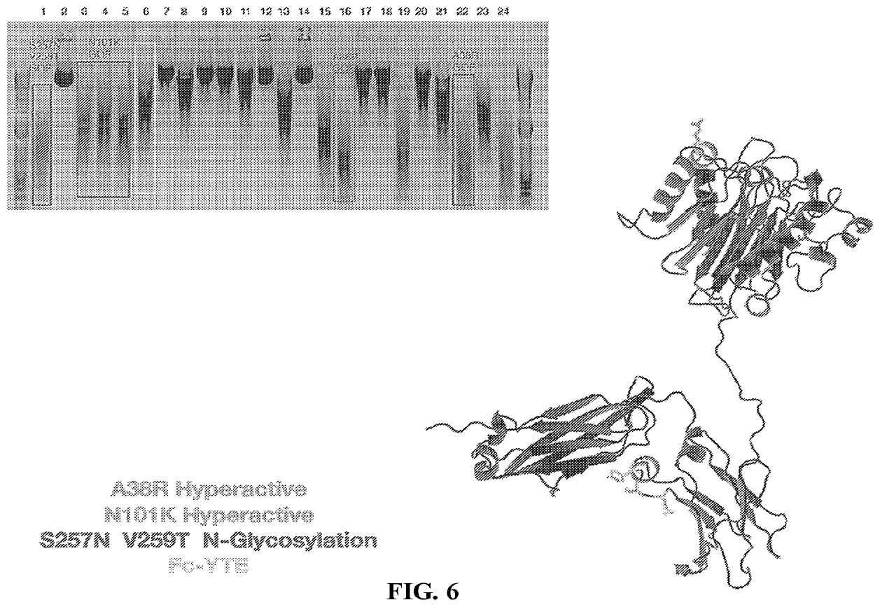

FIG. 6 illustrates non-limiting constructs of the disclosure, with certain contemplated point mutations highlighted. In certain embodiments, the construct lacks at least a portion of the DNAse1L3 nuclear localization domain.

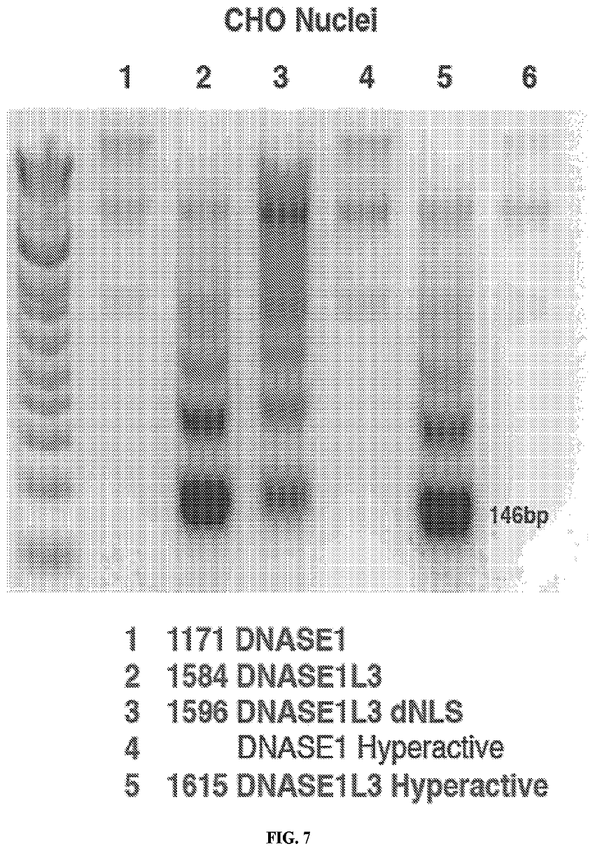

FIG. 7 illustrates a gel indicating that certain DNAse1L3 clones cleave chromatin, but that is not the case for certain DNAse1 clones.

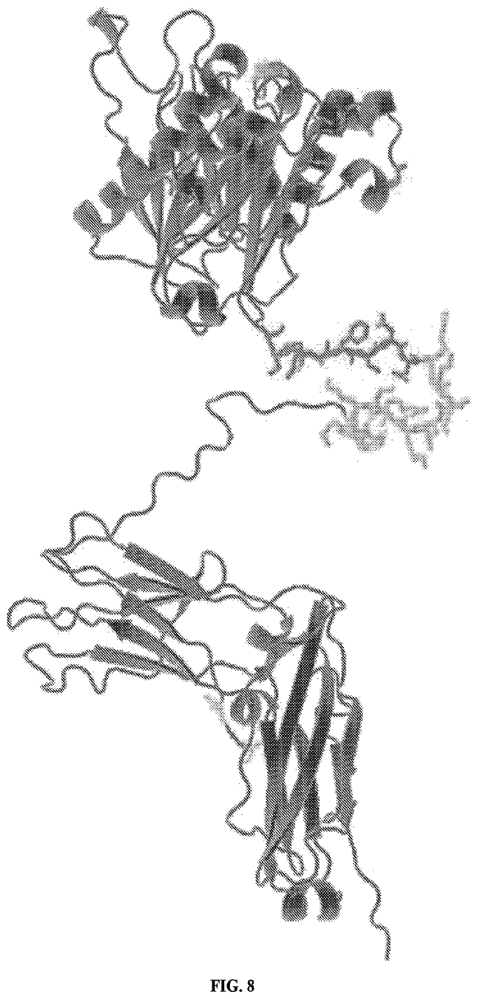

FIG. 8 illustrates a non-limiting construct of the disclosure. In certain embodiments, the DNAse1 polypeptide is fused with the C-terminus tail of DNAse1L3.

FIGS. 9A-9D illustrate certain aspects of production and purification of DNAse-Fc constructs.

FIG. 10 illustrates a non-limiting enzyme optimization pathway to be applied to NET degrading enzymes, as illustrated with an exemplary protein and/or polypeptide.

FIGS. 11A-11D illustrate selected results for optimization of NET degrading enzymes. FIG. 11A: Free (or plasmid) DNA in the blood was degraded by DNAse1. Histone associated DNA was degraded by DNAse1L3. FIG. 11B: PK Assay of optimized DNAse1 and DNAse1L3 constructs. Mice were injected with 1 mg/kg biologic; blood was withdrawn at various time points; exogenous plasmid or histone associated DNA is added; samples were incubated for 15 min.; degradation of DNA determined by agarose gels. FIGS. 11C-11D: PK of enzyme biologics determined in mice. Lanes 1-2: construct 1171; Lanes 3-4: construct 1671; Lanes 5-6: construct 1687; Lanes 7-8: Mock Injection. Constructs 1671 and 1687 readily degraded both plasmid (top) and chromatin DNA at 91 hours (FIG. 11C), and the activity persisted for 257 hours (FIG. 11D).

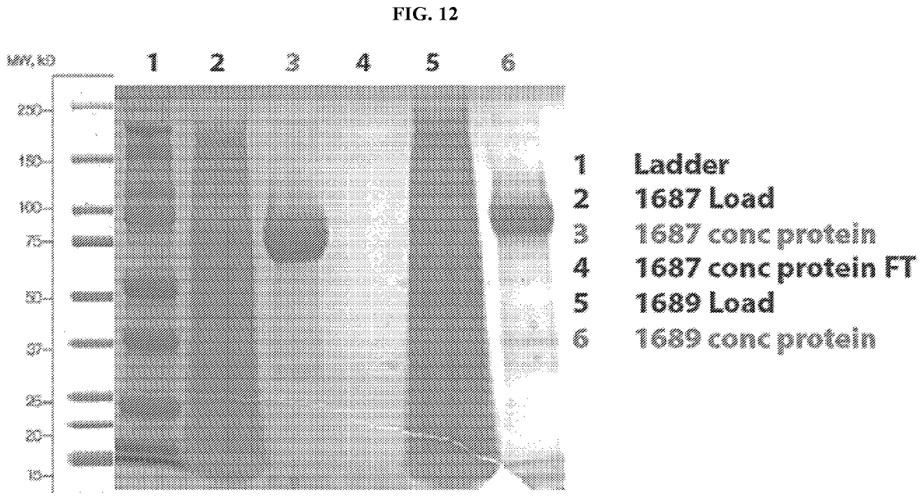

FIG. 12 illustrates certain aspects of production and purification of DNAse-Fc constructs.

### Enumerated Embodiments

The following exemplary embodiments are provided, the numbering of which is not to be construed as designating levels of importance:

Embodiment 1 provides a method of treating, ameliorating, and/or preventing inefficient NET hydrolysis (“NETolysis”) in a subject afflicted with a bacterial and/or viral infection, the method comprising administering to the subject a therapeutically effective amount of a construct comprising the amino acid sequence: Y—X1-LINKER-Fc-X2 (I), wherein: Y is a human DNAse1 polypeptide or a human DNAse1L3 polypeptide; X1 is a covalent bond, or X1 is the peptide of amino acid sequence RAFTNNRKSVSLKKRKKGNRS (SEQ ID NO:3) or a fragment thereof; LINKER is a chemical bond or a polypeptide comprising 1-100 amino acids; X2 is null, or X2 is the peptide of amino acid sequence RAFTNNRKSVSLKKRKKGNRS (SEQ ID NO:3) or a fragment thereof; Fc is the Fc domain of human IgG1.

Embodiment 2 provides a method of treating, ameliorating, and/or preventing systemic inflammation, organ damage, and/or sepsis in a subject afflicted with a bacterial and/or viral infection, the method comprising administering to the subject a therapeutically effective amount of a construct comprising the amino acid sequence: Y—X1-LINKER-Fc-X2 (I), wherein: Y is a human DNAse1 polypeptide or a human DNAse1L3 polypeptide; X1 is a covalent bond, or X1 is the peptide of amino acid sequence RAFTNNRKSVSLKKRKKGNRS (SEQ ID NO:3) or a fragment thereof; LINKER is a chemical bond or a polypeptide comprising 1-100 amino acids; X2 is null, or X2 is the peptide of amino acid sequence RAFTNNRKSVSLKKRKKGNRS (SEQ ID NO:3) or a fragment thereof; Fc is the Fc domain of human IgG1.

Embodiment 3 provides a method of treating, ameliorating, and/or preventing pathologic thrombosis in a subject afflicted with a bacterial and/or viral infection, the method comprising administering to the subject a therapeutically effective amount of a construct comprising the amino acid sequence: Y—X1-LINKER-Fc-X2 (I), wherein: Y is a human DNAse1 polypeptide or a human DNAse1L3 polypeptide; X1 is a covalent bond, or X1 is the peptide of amino acid sequence RAFTNNRKSVSLKKRKKGNRS (SEQ ID NO:3) or a fragment thereof; LINKER is a chemical bond or a polypeptide comprising 1-100 amino acids; X2 is null, or X2 is the peptide of amino acid sequence RAFTNNRKSVSLKKRKKGNRS (SEQ ID NO:3) or a fragment thereof; Fc is the Fc domain of human IgG1.

Embodiment 4 provides the method of any one of Embodiments 1-3, wherein the Fc comprises the amino acid sequence of SEQ ID NO:4.

Embodiment 5 provides the method of Embodiment 4, wherein at least one of C6 and C9 with respect to SEQ ID NO:4 is independently mutated to G or S.

Embodiment 6 provides the method of any one of Embodiments 4-5, wherein each one of C6 and C9 with respect to SEQ ID NO:4 is independently mutated to G or S.

Embodiment 7 provides the method of any one of Embodiments 4-6, comprising at least one of the following mutations with respect to SEQ ID NO:4: M32Y, S34T, T36E.

Embodiment 8 provides the method of any one of Embodiments 4-7, comprising each one of the following mutations with respect to SEQ ID NO:4: M32Y, S34T, T36E.

Embodiment 9 provides the method of any one of Embodiments 1-8, wherein the LINKER is a chemical bond or absent.

Embodiment 10 provides the method of any one of Embodiments 1-8, wherein the LINKER is a polypeptide comprising 1-100, 1-90, 1-80, 1-70, 1-60, 1-50, 1-40, 1-30, 1-20, 1-10, and/or 1-5 amino acids.

Embodiment 11 provides the method of any one of Embodiments 1-8 and 10, wherein the LINKER comprises GS and/or GSC.

Embodiment 12 provides the method of any one of Embodiments 1-9 and 10-11, wherein the LINKER comprises GGGGSGGGGS (SEQ ID NO:5), SSTMVRS (SEQ ID NO:40), and/or SSTMVGS (SEQ ID NO:41).

Embodiment 13 provides the method of any one of Embodiments 1-8 and 10-12, wherein the LINKER comprises ELKTPLGDTTHTXPRZPAPELLGGP (SEQ ID NO:6), wherein each occurrence of X is C, G, or S, and wherein each occurrence of Z is C, G, or S.

Embodiment 14 provides the method of any one of Embodiments 1-13, wherein X1 is a covalent bond.

Embodiment 15 provides the method of any one of Embodiments 1-13, wherein X1 is the peptide of amino acid sequence RAFTNNRKSVSLKKRKKGNRS (SEQ ID NO:3) or a fragment thereof.

Embodiment 16 provides the method of any one of Embodiments 1-15, wherein X2 is a covalent bond.

Embodiment 17 provides the method of any one of Embodiments 1-15, wherein X2 is the peptide of amino acid sequence RAFTNNRKSVSLKKRKKGNRS (SEQ ID NO:3) or a fragment thereof.

Embodiment 18 provides the method of any one of Embodiments 1-17, wherein the DNAse1 lacks at least a portion of residues 1-22 corresponding to SEQ ID NO:1.

Embodiment 19 provides the method of any one of Embodiments 1-18, wherein the DNAse1 lacks residues 1-22 corresponding to SEQ ID NO:1.

Embodiment 20 provides the method of any one of Embodiments 1-19, wherein the DNAse1 comprises at least one of the following mutations with respect to SEQ ID NO:1: Q31R, E35R, Y46H, Y46S, V88N, N96K, D109N, V111T, A136F, R148S, E149N, M186I, L208P, D220N, D250N, A252T, G262N, D265N, and L267T.

Embodiment 21 provides the method of any one of Embodiments 1-20, wherein the Fc comprises at least one of the following mutations with respect to SEQ ID NO:4: C6G, C6S, C9G, C9S, M32Y, S34T, and T36E.

Embodiment 22 provides the method of any one of Embodiments 1-21, which is selected from the group consisting of SEQ ID NOs:7-17 and 32-35.

Embodiment 23 provides the method of any one of Embodiments 1-17, wherein the DNAse1L3 lacks at least one of the following: residues 291-305 of SEQ ID NO:2; residues 296-304 of SEQ ID NO:2; residues 292-304 of SEQ ID NO:2; residues A-B of SEQ ID NO:2, wherein A ranges from 291 to 296 and B ranges from 304 to 305.

Embodiment 24 provides the method of any one of Embodiments 1-17 and 23, wherein the DNAse1L3 comprises at least one of the following mutations with respect to SEQ ID NO:2: E33R, M42T, V44H, V88T, N96K, A127N, V129T, K147S, D148N, L207P, D219N, and V254T.

Embodiment 25 provides the method of any one of Embodiments 1-17 and 23-24, wherein the Fc comprises at least one of the following mutations with respect to SEQ ID NO:4: C6G, C6S, C9G, C9S, M32Y, S34T, and T36E.

Embodiment 26 provides the method of any one of Embodiments 1-17 and 23-25, which is selected from the group consisting of SEQ ID NOs: 18-28 and 36-39.

Embodiment 27 provides the method of any one of Embodiments 1-17 and 23-26, wherein the construct is expressed in a mammalian cell.

Embodiment 28 provides the method of Embodiment 27, wherein the mammalian cell is stably transfected with human ST6 beta-galatosamide alpha-2,6-sialyltransferase (also known as ST6GAL1).

Embodiment 29 provides the method of any one of Embodiments 27-28, wherein the mammalian cell is grown in a cell culture supplemented with sialic acid and/or N-acetylmannosamine (also known as 1,3,4-O-Bu₃ManNAc).

Embodiment 30 provides the method of any one of Embodiments 1-29, wherein the construct is soluble.

Embodiment 31 provides the method of any one of Embodiments 1-30, wherein the virus is a coronavirus.

Embodiment 32 provides the method of Embodiment 31, wherein the coronavirus is SARS-Cov and/or SARS-Cov-2.

Embodiment 33 provides the method of any one of Embodiments 3-32, wherein the thrombosis leads to stroke or makes the subject susceptible to stroke.

Embodiment 34 provides the method of any one of Embodiments 1-33, wherein in the DNAse1 and/or DNAse1L3 precursor construct the signal peptide sequence is conjugated to the N-terminus of the DNAse1 and/or DNAse1L3 polypeptide.

Embodiment 35 provides the method of any one of Embodiments 1-34, wherein the construct is a secreted product of a DNAse1 and/or DNAse1L3 precursor construct expressed in a mammalian cell, wherein the DNAse1 and/or DNAse1L3 precursor construct comprises a signal peptide sequence and a DNAse1 and/or DNAse1L3 polypeptide, wherein the DNAse1 and/or DNAse1L3 precursor construct undergoes proteolytic processing to yield the DNAse1 and/or DNAse1L3 construct.

Embodiment 36 provides the method of any one of Embodiments 1-35, wherein the construct is administered acutely or chronically to the subject.

Embodiment 37 provides the method of any one of Embodiments 1-36, wherein the construct is administered locally, regionally, parenterally, or systemically to the subject.

Embodiment 38 provides the method of any one of Embodiments 1-37, wherein the construct, or its precursor construct, is delivered on an encoded vector to the subject, wherein the vector encodes the construct or precursor construct, which is transcribed and translated from the vector upon administration of the vector to the subject.

Embodiment 39 provides the method of any one of Embodiments 1-38, wherein the construct is administered to the subject by at least one route selected from the group consisting of subcutaneous, oral, aerosol, inhalational, rectal, vaginal, transdermal, subcutaneous, intranasal, buccal, sublingual, parenteral, intrathecal, intragastrical, ophthalmic, pulmonary, and topical.

Embodiment 40 provides the method of any one of Embodiments 1-39, wherein the construct is administered to the subject as a pharmaceutical composition further comprising at least one pharmaceutically acceptable carrier.

Embodiment 41 provides the method of any one of Embodiments 1-40, wherein the construct comprises at least one of the following:
(a) a homodimeric construct comprising two independently selected constructs (I), wherein each Y is an independently selected human DNAse1 polypeptide;
(b) a homodimeric construct comprising two independently selected constructs (I), wherein each Y is an independently selected human DNAse1L3 polypeptide; and/or
(c) a heterodimeric construct comprising two independently selected constructs (I), wherein the Y in one of the two (I) is a human DNAse1 polypeptide and the Y in the other (I) is a human DNAse1L3 polypeptide.

Embodiment 42 provides the method of any one of Embodiments 1-41, wherein the subject is a mammal.

Embodiment 43 provides the method of Embodiment 42, wherein the mammal is human.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1. A method of:
(a) treating, ameliorating, or preventing inefficient NET hydrolysis (“NETolysis”) in a subject afflicted with a bacterial or viral infection,
(b) treating, ameliorating, or preventing systemic inflammation, organ damage, or sepsis in a subject afflicted with a bacterial or viral infection, or
(c) treating, ameliorating, or preventing pathologic thrombosis in a subject afflicted with a bacterial or viral infection;
the method comprising administering to the subject a therapeutically effective amount of a construct comprising the amino acid sequence:
Y—X1-LINKER-Fc-X2 (I)
wherein:
Y is a human DNAse1 polypeptide or a human DNAse1L3 polypeptide;
X1 is a covalent bond, or X1 is the peptide of amino acid sequence RAFTNNRKSVSLKKRKKGNRS (SEQ ID NO:3) or a fragment thereof;
LINKER is a chemical bond or a polypeptide comprising 1-100 amino acids;
X2 is null, or X2 is the peptide of amino acid sequence RAFTNNRKSVSLKKRKKGNRS (SEQ ID NO:3) or a fragment thereof;
Fc is the Fc domain of human IgG1.

2-3. (canceled)

4. The method of claim 1, wherein the Fc comprises the amino acid sequence of SEQ ID NO:4.

5. The method of claim 4, wherein at least one of C6 and C9 with respect to SEQ ID NO:4 is independently mutated to G or S, optionally wherein each one of C6 and C9 with respect to SEQ ID NO:4 is independently mutated to G or S.

6. (canceled)

7. The method of claim 4, comprising at least one of the following mutations with respect to SEQ ID NO:4: M32Y, S34T, T36E, optionally comprising each one of the following mutations with respect to SEQ ID NO:4: M32Y, S34T, T36E.

8. (canceled)

9. The method of claim 1, wherein at least one of the following applies:
(a) the LINKER is a chemical bond or absent;
(b) the LINKER is a polypeptide comprising 1 100, 1-90, 1-80, 1-70, 1-60, 1-50, 1-40, 1-30, 1-20, 1-10, or 1-5 amino acids;
(c) the LINKER comprises GS or GSC;
(d) the LINKER comprises GGGGSGGGGS (SEQ ID NO:5), SSTMVRS (SEQ ID NO:40), or SSTMVGS (SEQ ID NO:41);
(e) the LINKER comprises ELKTPLGDTTHTXPRZPAPELLGGP (SEQ ID NO:6), wherein each occurrence of X is C, G, or S, and wherein each occurrence of Z is C, G, or S.

10-13. (canceled)

14. The method of claim 1, wherein at least one of the following applies:
(a) X1 is a covalent bond;
(b) X1 is the peptide of amino acid sequence RAFTNNRKSVSLKKRKKGNRS (SEQ ID NO:3) or a fragment thereof;
(c) X2 is a covalent bond;
(d) X2 is the peptide of amino acid sequence RAFTNNRKSVSLKKRKKGNRS (SEQ ID NO:3) or a fragment thereof.

15-17. (canceled)

18. The method of claim 1, wherein the DNAse1 lacks at least a portion of residues 1-22 corresponding to SEQ ID NO:1, optionally wherein the DNAse1 lacks residues 1-22 corresponding to SEQ ID NO:1.

19. (canceled)

20. The method of claim 1, wherein the DNAse1 comprises at least one of the following mutations with respect to SEQ ID NO:1: Q31R, E35R, Y46H, Y46S, V88N, N96K, D109N, V111T, A136F, R148S, E149N, M186I, L208P, D220N, D250N, A252T, G262N, D265N, and L267T.

21. The method of claim 1, wherein the Fc comprises at least one of the following mutations with respect to SEQ ID NO:4: C6G, C6S, C9G, C9S, M32Y, S34T, and T36E.

22. The method of claim 1, which is selected from the group consisting of SEQ ID NOs:7-17 and 32-35.

23. The method of claim 1, wherein at least one the following applies:
(a) the DNAse1L3 lacks at least one of the following: residues 291-305 of SEQ ID NO:2; residues 296-304 of SEQ ID NO:2; residues 292-304 of SEQ ID NO:2; residues A-B of SEQ ID NO:2, wherein A ranges from 291 to 296 and B ranges from 304 to 305;
(b) the DNAse1L3 comprises at least one of the following mutations with respect to SEQ ID NO:2: E33R, M42T, V44H, V88T, N96K, A127N, V129T, K147S, D148N, L207P, D219N, and V254T.

24. (canceled)

25. The method of claim 1, wherein the Fc comprises at least one of the following mutations with respect to SEQ ID NO:4: C6G, C6S, C9G, C9S, M32Y, S34T, and T36E.

26. The method of claim 1, which is selected from the group consisting of SEQ ID NOs: 18-28 and 36-39.

27. The method of claim 1, wherein the construct is expressed in a mammalian cell.

28. The method of claim 27, wherein at least one of the following applies:
(a) the mammalian cell is stably transfected with human ST6 beta-galatosamide alpha-2,6-sialyltransferase (also known as ST6GAL1);
(b) the mammalian cell is grown in a cell culture supplemented with sialic acid and/or N-acetylmannosamine (also known as 1,3,4-O-Bu₃ManNAc).

29. (canceled)

30. The method of claim 1, wherein the construct is soluble.

31. The method of claim 1, wherein the virus is a coronavirus, optionally wherein the coronavirus is SARS-Cov or SARS-Cov-2.

32. (canceled)

33. The method of claim 1, wherein the thrombosis leads to stroke or makes the subject susceptible to stroke.

34. The method of claim 1, wherein in the DNAse1 or DNAse1L3 precursor construct the signal peptide sequence is conjugated to the N-terminus of the DNAse1 or DNAse1L3 polypeptide.

35. The method of claim 1, wherein the construct is a secreted product of a DNAse1 or DNAse1L3 precursor construct expressed in a mammalian cell, wherein the DNAse1 or DNAse1L3 precursor construct comprises a signal peptide sequence and a DNAse1 or DNAse1L3 polypeptide, wherein the DNAse1 or DNAse1L3 precursor construct undergoes proteolytic processing to yield the DNAse1 or DNAse1L3 construct.

36. The method of claim 1, wherein at least one of the following applies:
(a) the construct is administered acutely or chronically to the subject;
(b) the construct is administered locally, regionally, parenterally, or systemically to the subject;
(c) the construct, or its precursor construct, is delivered on an encoded vector to the subject, wherein the vector encodes the construct or precursor construct, which is transcribed and translated from the vector upon administration of the vector to the subject;
(d) the construct is administered to the subject by at least one route selected from the group consisting of subcutaneous, oral, aerosol, inhalational, rectal, vaginal, transdermal, subcutaneous, intranasal, buccal, sublingual, parenteral, intrathecal, intragastrical, ophthalmic, pulmonary, and topical;
(e) the construct is administered to the subject as a pharmaceutical composition further comprising at least one pharmaceutically acceptable carrier.

37-40. (canceled)

41. The method of claim 1, wherein the construct comprises at least one of the following:
(a) a homodimeric construct comprising two independently selected constructs (I), wherein each Y is an independently selected human DNAse1 polypeptide;
(b) a homodimeric construct comprising two independently selected constructs (I), wherein each Y is an independently selected human DNAse1L3 polypeptide;
(c) a heterodimeric construct comprising two independently selected constructs (I), wherein the Y in one of the two (I) is a human DNAse1 polypeptide and the Y in the other (I) is a human DNAse1L3 polypeptide.

42. The method of claim 1, wherein the subject is a mammal, optionally the mammal is human.

43. (canceled)


SEQUENCE LISTING (43 sequences)

SEQ ID NO: 1 - protein, 282 aa, Homo sapiens [sequence omitted]

SEQ ID NO: 2 - protein, 305 aa, Homo sapiens
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRS

SEQ ID NO: 3 - protein, 21 aa, Artificial Sequence
  Chemically synthesized
RAFTNNRKSVSLKKRKKGNRS

SEQ ID NO: 4 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 10 aa, Artificial Sequence
  Chemically synthesized
GGGGSGGGGS

SEQ ID NO: 6 - protein, 25 aa, Artificial Sequence
  Chemically synthesized
  MOD_RES (13)..(13): Cys, Gly, or Ser
  MOD_RES (16)..(16): Cys, Gly, or Ser
ELKTPLGDTTHTXPRXPAPELLGGP

SEQ ID NO: 7 - protein, 511 aa, Artificial Sequence
  Chemically synthesized
MRGMKLLGALLALAALLQGAVSLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 511 aa, Artificial Sequence
  Chemically synthesized
MRGMKLLGALLALAALLQGAVSLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKGSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 511 aa, Artificial Sequence
  Chemically synthesized
MRGMKLLGALLALAALLQGAVSLKIAAFNIQTFGRTKMSNATLVSYIVQILSRYDIALVQEVRDSHLTAVGKLLDNLNQDAPDTYHYNVSEPLGRNSYKERYLFVYRPNQTSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYNLSNQTAQAISDHYPVEVMLKGSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 553 aa, Artificial Sequence
  Chemically synthesized
MRGMKLLGALLALAALLQGAVSLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKRAFTNNRKSVSLKKRKKGNRSGSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKRAFTNNRKSVSLKKRKKGNRS

SEQ ID NO: 11 - protein, 532 aa, Artificial Sequence
  Chemically synthesized
MRGMKLLGALLALAALLQGAVSLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKGSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKRAFTNNRKSVSLKKRKKGNRS

SEQ ID NO: 12 - protein, 532 aa, Artificial Sequence
  Chemically synthesized
MRGMKLLGALLALAALLQGAVSLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKRAFTNNRKSVSLKKRKKGNRSGSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 519 aa, Artificial Sequence
  Chemically synthesized
MRGMKLLGALLALAALLQGAVSLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKGGGGSGGGGSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 534 aa, Artificial Sequence
  Chemically synthesized
  MOD_RES (295)..(295): Cys, Gly, or Ser
  MOD_RES (298)..(298): Cys, Gly, or Ser
MRGMKLLGALLALAALLQGAVSLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKELKTPLGDTTHTXPRXPAPELLGGPDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 512 aa, Artificial Sequence
  Chemically synthesized
MRGMKLLGALLALAALLQGAVSLKIAAFNIRTFGETKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLQGAVVPDSALPFNFQAAYGLSDQLAQAISDHYPVEVMLKGSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 512 aa, Artificial Sequence
  Chemically synthesized
MRGMKLLGALLALAALLQGAVSLKIAAFNIRTFGETKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLQGAVVPDSALPFNFQAAYGLSNQTAQAISDHYPVEVMLKGSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 512 aa, Artificial Sequence
  Chemically synthesized
MRGMKLLGALLALAALLQGAVSLKIAAFNIRTFGETKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLQGAVVPNSTLPFNFQAAYGLSDQLAQAISDHYPVEVMLKGSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 534 aa, Artificial Sequence
  Chemically synthesized
MSRELAPLLLLLLSIHSALAMRICSFNVRSFGESKQEDKNAMDVIVKVIKRCDIILVMEIKDSNNRICPILMEKLNRNSRRGITYNYVISSRLGRKTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 534 aa, Artificial Sequence
  Chemically synthesized
MSRELAPLLLLLLSIHSALAMRICSFNVRSFGESKQEDKNAMDVIVKVIKRCDIILVMEIKDSNNRICPILMEKLNRNSRRGITYNYVISSRLGRKTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSGSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 555 aa, Artificial Sequence
  Chemically synthesized
MSRELAPLLLLLLSIHSALAMRICSFNVRSFGESKQEDKNAMDVIVKVIKRCDIILVMEIKDSNNRICPILMEKLNRNSRRGITYNYVISSRLGRKTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSGSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKRAFTNNRKSVSLKKRKKGNRS

SEQ ID NO: 21 - protein, 576 aa, Artificial Sequence
  Chemically synthesized
MSRELAPLLLLLLSIHSALAMRICSFNVRSFGESKQEDKNAMDVIVKVIKRCDIILVMEIKDSNNRICPILMEKLNRNSRRGITYNYVISSRLGRKTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSRAFTNNRKSVSLKKRKKGNRSGSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKRAFTNNRKSVSLKKRKKGNRS

SEQ ID NO: 22 - protein, 555 aa, Artificial Sequence
  Chemically synthesized
MSRELAPLLLLLLSIHSALAMRICSFNVRSFGESKQEDKNAMDVIVKVIKRCDIILVMEIKDSNNRICPILMEKLNRNSRRGITYNYVISSRLGRKTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSRAFTNNRKSVSLKKRKKGNRSGSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 542 aa, Artificial Sequence
  Chemically synthesized
MSRELAPLLLLLLSIHSALAMRICSFNVRSFGESKQEDKNAMDVIVKVIKRCDIILVMEIKDSNNRICPILMEKLNRNSRRGITYNYVISSRLGRKTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSGGGGSGGGGSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 557 aa, Artificial Sequence
  Chemically synthesized
  MOD_RES (318)..(318): Cys, Gly, or Ser
  MOD_RES (321)..(321): Cys, Gly, or Ser
  MOD_RES (336)..(336): Cys, Gly, or Ser
MSRELAPLLLLLLSIHSALAMRICSFNVRSFGESKQEDKNAMDVIVKVIKRCDIILVMEIKDSNNRICPILMEKLNRNSRRGITYNYVISSRLGRKTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSELKTPLGDTTHTXPRXPAPEFLGGPDKTHTXPPZPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 519 aa, Artificial Sequence
  Chemically synthesized
  MOD_RES (298)..(298): Cys, Gly, or Ser
  MOD_RES (301)..(301): Cys, Gly, or Ser
MSRELAPLLLLLLSIHSALAMRICSFNVRSFGESKQEDKNAMDVIVKVIKRCDIILVMEIKDSNNRICPILMEKLNRNSRRGITYNYVISSRLGRKTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSGSDKTHTXPPZPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 535 aa, Artificial Sequence
  Chemically synthesized
MSRELAPLLLLLLSIHSALAMRICSFNVRSFGRSKQEDKNATDVIVKVIKRCDIILVMEIKDSNNRICPILMEKLNRNSRRGITYNYVISSRLGRKTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSGSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 535 aa, Artificial Sequence
  Chemically synthesized
MSRELAPLLLLLLSIHSALAMRICSFNVRSFGRSKQEDKNATDVIVKVIKRCDIILVMEIKDSNNRICPILMEKLNRNSRRGITYNYVISSRLGRKTYKEQYAFLYKEKLVSVKRSYHYHDYQDGDNDTFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSGSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 535 aa, Artificial Sequence
  Chemically synthesized
MSRELAPLLLLLLSIHSALAMRICSFNVRSFGRSKQEDKNATDVIVKVIKRCDIILVMEIKDSNNRICPILMEKLNRNSRRGITYNYTISSRLGRKTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSGSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 283 aa, Mus musculus
MRYTGLMGTLLTLVNLLQLAGTLRIAAFNIRTFGETKMSNATLSVYFVKILSRYDIAVIQEVRDSHLVAVGKLLDELNRDKPDTYRYVVSEPLGRKSYKEQYLFVYRPDQVSILDSYQYDDGCECGNDTFSREPAIVKFFSPYTEVQEFAIVPLHAAPTEAVSEIDALYDVYLDVWQKWGLEDIMFMGDFNAGCSYVTSSQWSSIRLRTSPIFQWLIPDSADTTVTSTHCAYDRIVVAGALLQAAVVPNSAVPFDFQAEYGLSNQLAEAISDHYPVEVTLRKI

SEQ ID NO: 30 - protein, 310 aa, Mus musculus
MSLHPASPRLASLLLFILALHDTLALRLCSFNVRSFGASKKENHEAMDIIVKIIKRCDLILLMEIKDSSNNICPMLMEKLNGNSRRSTTYNYVISSRLGRNTYKEQYAFVYKEKLVSVKTKYHYHDYQDGDTDVFSREPFVVWFHSPFTAVKDFVIVPLHTTPETSVKEIDELVDVYTDVRSQWKTENFIFMGDFNAGCSYVPKKAWQNIRLRTDPKFVWLIGDQEDTTVKKSTSCAYDRIVLCGQEIVNSVVPRSSGVFDFQKAYDLSEEEALDVSDHFPVEFKLQSSRAFTNNRKSVSLKKRKKGNRS

SEQ ID NO: 31 - protein, 222 aa, Mus musculus
GCKPCICTVPEVSSVFIFPPKPKDVLYITLEPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 32 - protein, 512 aa, Artificial Sequence
  Chemically synthesized
MRYTGLMGTLLTLVNLLQLAGTLRIAAFNIRTFGETKMSNATLSVYFVKILSRYDIAVIQEVRDSHLVAVGKLLDELNRDKPDTYRYVVSEPLGRKSYKEQYLFVYRPDQVSILDSYQYDDGCECGNDTFSREPAIVKFFSPYTEVQEFAIVPLHAAPTEAVSEIDALYDVYLDVWQKWGLEDIMFMGDFNAGCSYVTSSQWSSIRLRTSPIFQWLIPDSADTTVTSTHCAYDRIVVAGALLQAAVVPNSAVPFDFQAEYGLSNQLAEAISDHYPVEVTLRKISSTMVRSGCKPCICTVPEVSSVFIFPPKPKDVLYITLEPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 33 - protein, 513 aa, Artificial Sequence
  Chemically synthesized
MRYTGLMGTLLTLVNLLQLAGTLRIAAFNIRTFGETKMSNATLSVYFVKILSRYDIAVIQEVRDSHLVAVGKLLDELNRDKPDTYRYVVSEPLGRKSYKEQYLFVYRPDQVSILDSYQYDDGCEPCGNDTFSREPFIVKFFSPYTEVQEFAIVPLHAAPTEAVSEIDALYDVYLDVWQKWGLEDIMFMGDFNAGCSYVTSSQWSSIRLRTSPIFQWLIPDSADTTVTSTHCAYDRIVVAGALLQAAVVPNSAVPFDFQAEYGLSNQLAEAISDHYPVEVTLRKISSTMVGSGCKPCICTVPEVSSVFIFPPKPKDVLYITLEPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 34 - protein, 513 aa, Artificial Sequence
  Chemically synthesized
MRYTGLMGTLLTLVNLLQLAGTLRIAAFNIRTFGRTKMSNATLSVYFVKILSRYDIAVIQEVRDSHLVAVGKLLDELNRDKPDTYRYNVSEPLGRKSYKEQYLFVYRPDQVSILDSYQYDDGCEPCGNDTFSREPAIVKFFSPYTEVQEFAIVPLHAAPTEAVSEIDALYDVYLDVWQKWGLEDIMFMGDFNAGCSYVTSSQWSSIRLRTSPIFQWLIPDSADTTVTSTHCAYDRIVVAGALLQAAVVPNSAVPFDFQAEYGLSNQLAEAISDHYPVEVTLRKISSTMVGSGCKPCICTVPEVSSVFIFPPKPKDVLYITLEPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 35 - protein, 513 aa, Artificial Sequence
  Chemically synthesized
MRYTGLMGTLLTLVNLLQLAGTLRIAAFNIRTFGRTKMSNATLSVYFVKILSRYDIAVIQEVRDSHLVAVGKLLDELNRDKPDTYRYNVSEPLGRKSYKEQYLFVYRPDQVSILDSYQYDDGCEPCGNDTFSREPAIVKFFSPYTEVQEFAIVPLHAAPTEAVSEIDALYDVYLDVWQKWGLEDIMFMGDFNAGCSYVTSSQWSSIRLRTSPIFQWLIPDSADTTVTSTHCAYDRIVVAGALLQAAVVPNSAVPFDFQAEYGLSNQTAEAISDHYPVEVTLRKISSTMVGSGCKPCICTVPEVSSVFIFPPKPKDVLYITLEPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 36 - protein, 539 aa, Artificial Sequence
  Chemically synthesized
MSLHPASPRLASLLLFILALHDTLALRLCSFNVRSFGASKKENHEAMDIIVKIIKRCDLILLMEIKDSSNNICPMLMEKLNGNSRRSTTYNYVISSRLGRNTYKEQYAFVYKEKLVSVKTKYHYHDYQDGDTDVFSREPFVVWFHSPFTAVKDFVIVPLHTTPETSVKEIDELVDVYTDVRSQWKTENFIFMGDFNAGCSYVPKKAWQNIRLRTDPKFVWLIGDQEDTTVKKSTSCAYDRIVLCGQEIVNSVVPRSSGVFDFQKAYDLSEEEALDVSDHFPVEFKLQSSRAFTNNRKSVSLKKRKKGNRSSSTMVGSGCKPCICTVPEVSSVFIFPPKPKDVLYITLEPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 37 - protein, 519 aa, Artificial Sequence
  Chemically synthesized
MSLHPASPRLASLLLFILALHDTLALRLCSFNVRSFGASKKENHEAMDIIVKIIKRCDLILLMEIKDSSNNICPMLMEKLNGNSRRSTTYNYVISSRLGRNTYKEQYAFVYKEKLVSVKTKYHYHDYQDGDTDVFSREPFVVWFHSPFTAVKDFVIVPLHTTPETSVKEIDELVDVYTDVRSQWKTENFIFMGDFNAGCSYVPKKAWQNIRLRTDPKFVWLIGDQEDTTVKKSTSCAYDRIVLCGQEIVNSVVPRSSGVFDFQKAYDLSEEEALDVSDHFPVEFKLQSSRAFTNNRSGCKPCICTVPEVSSVFIFPPKPKDVLYITLEPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 38 - protein, 539 aa, Artificial Sequence
  Chemically synthesized
MSLHPASPRLASLLLFILALHDTLALRLCSFNVRSFGRSKKENHEAMDIIVKIIKRCDLILLMEIKDSSNNICPMLMEKLNGNSRRSTTYNYVISSRLGRKTYKEQYAFVYKEKLVSVKTKYHYHDYQDGDTDVFSREPFVVWFHSPFTAVKDFVIVPLHTTPETSVKEIDELVDVYTDVRSQWKTENFIFMGDFNAGCSYVPKKAWQNIRLRTDPKFVWLIGDQEDTTVKKSTSCAYDRIVLCGQEIVNSVVPRSSGVFDFQKAYDLSEEEALDVSDHFPVEFKLQSSRAFTNNRKSVSLKKRKKGNRSSSTMVGSGCKPCICTVPEVSSVFIFPPKPKDVLYITLEPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 39 - protein, 539 aa, Artificial Sequence
  Chemically synthesized
MSLHPASPRLASLLLFILALHDTLALRLCSFNVRSFGRSKKENHEAMDIIVKIIKRCDLILLMEIKDSSNNICPMLMEKLNGNSRRSTTYNYVISSRLGRKTYKEQYAFVYKEKLVSVKTKYHYHDYQDGDTDVFSREPFVVWFHSPFTAVKDFVIVPLHTTPETSVKEIDELVDVYTDVRSQWKTENFIFMGDFNAGCSYVPKKAWQNIRLRTDPKFVWLIGDQEDTTVKKSTSCAYDRIVLCGQEIVNSVVPRSNGTFDFQKAYDLSEEEALDVSDHFPVEFKLQSSRAFTNNRKSVSLKKRKKGNRSSSTMVGSGCKPCICTVPEVSSVFIFPPKPKDVLYITLEPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 40 - protein, 7 aa, Artificial Sequence
  Chemically synthesized
SSTMVRS

SEQ ID NO: 41 - protein, 7 aa, Artificial Sequence
  Chemically synthesized
SSTMVGS

SEQ ID NO: 42 - protein, 507 aa, Artificial Sequence
  Chemically synthesized
MGTLLTLVNLLQLAGTLRIAAFNIRTFGETKMSNATLSVYFVKILSRYDIAVIQEVRDSHLVAVGKLLDELNRDKPDTYRYVVSEPLGRKSYKEQYLFVYRPDQVSILDSYQYDDGCEPCGNDTFSREPAIVKFFSPYTEVQEFAIVPLHAAPTEAVSEIDALYDVYLDVWQKWGLEDIMFMGDFNAGCSYVTSSQWSSIRLRTSPIFQWLIPDSADTTVTSTHCAYDRIVVAGALLQAAVVPNSAVPFDFQAEYGLSNQLAEAISDHYPVEVTLRKISSTMVGSGCKPCICTVPEVSSVFIFPPKPKDVLYITLEPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 43 - protein, 530 aa, Artificial Sequence
  Chemically synthesized
LASLLLFILALHDTLALRLCSFNVRSFGRSKKENHEAMDIIVKIIKRCDLILLMEIKDSSNNICPMLMEKLNGNSRRSTTYNYVISSRLGRKTYKEQYAFVYKEKLVSVKTKYHYHDYQDGDTDVFSREPFVVWFHSPFTAVKDFVIVPLHTTPETSVKEIDELVDVYTDVRSQWKTENFIFMGDFNAGCSYVPKKAWQNIRLRTDPKFVWLIGDQEDTTVKKSTSCAYDRIVLCGQEIVNSVVPRSSGVFDFQKAYDLSEEEALDVSDHFPVEFKLQSSRAFTNNRKSVSLKKRKKGNRSSSTMVGSGCKPCICTVPEVSSVFIFPPKPKDVLYITLEPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK